(19) United States
(12) Patent Application Publication
(10) Pub. No.: US 2020/0131196 A1
(43) Pub. Date: Apr. 30, 2020

(54) Rapamycin Analog

(71) Applicant: Mount Tam Therapeutics, Inc., Burlington, VT (US)

(72) Inventors: Timothy S. Powers, Novato, CA (US); Bryan F. Cox, Spring Grove, IL (US); Richard B. Marshak, Burlington, VT (US)

(73) Assignee: Mount Tam Therapeutics, Inc., Burlington, VT (US)

(21) Appl. No.: 16/484,665

(22) PCT Filed: Feb. 9, 2018
(86) PCT No.: PCT/US2018/017570

§ 371 Date: Aug. 8, 2019

Related U.S. Application Data
(60) Provisional application No. 62/457,676, filed on Feb. 10, 2017.

(51) Int. Cl. C07D 498/18; A61P 35/00

(52) U.S. Cl. CPC C07D 498/18; A61K 45/06; A61P 35/00

(57) ABSTRACT

The present invention relates to a novel rapamycin analogue (e.g., of Formula I or Formula II), mixtures, methods for its production, and its use in cancer therapy (e.g., prevention and/or treatment).

### RELATED APPLICATIONS

This application is a 35 U.S.C. § 371 national stage application of International Application No. PCT/US2018/017570, filed Feb. 9, 2018, and claims priority to U.S. Ser. No. 62/457,676 filed Feb. 10, 2017, both of which are hereby incorporated in their entirety into this application.

### BACKGROUND OF THE DISCLOSURE

Rapamycin (sirolimus) is a polyketide that is used to coat coronary stents and prevent organ transplant rejection. The art also suggests that rapamycin and rapamycin analogs can be used to treat lymphangioleiomyomatosis, pulmonary inflammation (U.S. Pat. No. 5,080,999), insulin dependent diabetes (U.S. Pat. No. 5,362,718 citing Fifth Int. Conf. Inflamm. Res. Assoc. 121 (Abstract), (1990)), certain coronary diseases (Morris, (1992) Heart Lung Transplant 11:197), leukemia and lymphoma (European Patent Application 0 525 960), and ocular inflammation (European Patent Application 0 532 862).

Rapamycin (FIG. 4) is produced by Streptomyces hygroscopicus NRRL 5491 (Sehgal et al., 1975; Vezina et al., 1975; U.S. Pat. Nos. 3,929,992; 3,993,749). For the purpose of this disclosure, rapamycin is described by the numbering convention of McAlpine et al. (1991) (see FIG. 3) in preference to the numbering conventions of Findlay et al. (1980) or Chemical Abstracts (11th Cumulative Index, 1982-1986 p60719CS).

U.S. Pat. No. 5,362,718 discloses acylated prodrugs of rapamycin.

Rapamycin and its commercially available analogs Temsirolimus and Everolimus inhibit activation of T cells and B cells by binding to mTOR which, among other things, reduces the production of interleukin-2. mTOR is the catalytic subunit of two structurally distinct complexes: mTORC1 and mTORC2 (Wang et al. (2006) Journal of Biological Chemistry, 281: 24293-303). mTORC1 and mTORC2 localize to different subcellular compartments, which affects their activation and function.

Scientific evidence suggests that mTORC1 functions as a sensor of cellular nutritional and energy status and has a role in the regulation of protein synthesis (Hay et al. (2004) Genes & Development 18: 1926-45; Kim et al. (2002) Cell, 110: 163-75). The activity of mTORC1 is regulated by rapamycin analogs, insulin, growth factors, phosphatidic acid, some amino acids and amino acid derivatives, mechanical stimuli, and oxidative stress.

Scientific evidence suggests that mTORC2 functions an important regulator of the actin cytoskeleton through its stimulation of F-actin stress fibers, paxillin, RhoA, Rac1, Cdc42, and protein kinase Ca (Sarbassov et al. (2004) Current Biology 14:1296-302). mTORC2 also affecting metabolism and survival apparently through phosphorylation of Akt/PKB (Betz et al. (2013) PNAS 110: 12526-34). Akt phosphorylation by mTORC2 interacts with PDK1 and leads to full Akt activation (Sarbassov et al. (2005) Science 307: 1098-101; Stephens et al. (1998) Science 279: 710-4). In addition, mTORC2 is capable of activating IGF-IR and InsR (Yin et al. (2016) Cell Research 26: 46-65).

While not intending to be bound be theory, we believe that rapamycin-like polyketide inhibitors of mTOR having a more balanced (e.g., less selective) ability to inhibit mTORC1 and mTORC2 are preferred for the treatment of certain cancers because inhibition of both mTORC1 and mTORC2 disables an escape mechanism through which drug resistance can develop.

Rapamycin analogs (including Rapamycin) have significant therapeutic value (Huang et al, 2003). These polyketides are a potent inhibitor of the mammalian target of rapamycin (mTOR), a serine-threonine kinase downstream of the phosphatidylinositol 3-kinase (PI3K)/Akt (protein kinase B) signaling pathway that mediates cell survival and proliferation. This inhibitory activity is gained after rapamycin binds to the immunophilin FK506 binding protein 12 (FKBP12) (Dumont, F. J. and Q. X. Su, 1995). In T cells, rapamycin inhibits signaling from the IL-2 receptor and subsequent autoproliferation of the T cells resulting in immunosuppression. Rapamycin is marketed as an immunosuppressant for the treatment of organ transplant patients to prevent graft rejection (Huang et al, 2003). In addition to immunosuppression, rapamycin has found therapeutic application in cancer (Vignot et al, 2005).

Rapamycin and many rapamycin analogs have disadvantages including inducement of hyperlipidemia, cellular efflux mediated by P-glycoprotein (“P-gp”; LaPlante et al, 2002, Crowe et al, 1999) and other efflux mechanisms which pumps the compound out of cells and tends to decrease effectiveness of administered drug compound and presents challenges to the treatment of multidrug resistant cancer. Hepatic first pass loss of rapamycin is also high, which contributes further to its low oral bioavailability. The low oral bioavailability of rapamycin causes significant inter-individual variability resulting in inconsistent therapeutic outcome and difficulty in clinical management (Kuhn et al, 2001, Crowe et al, 1999).

A wide range of synthesized rapamycin analogues using the chemically available sites of the molecule are known in the art. Chemically available sites on the molecule for derivatization or replacement are known in the art to include, for example, C40 and C28 hydroxyl groups (e.g. U.S. Pat. Nos. 5,665,772; 5,362,718); C39 and C16 methoxy groups (e.g. WO 96/41807; U.S. Pat. No. 5,728,710); C32, C26 and C9 keto groups (e.g. U.S. Pat. Nos. 5,378,836; 5,138,051; 5,665,772); hydrogenation at C17, C19 and/or C21 (e.g. U.S. Pat. Nos. 5,391,730; 5,023,262); and/or the formation of oximes at C32, C40 and/or C28, (e.g., U.S. Pat. Nos. 5,563,145, 5,446,048). Analogues exhibiting resistance to metabolic attack (e.g. U.S. Pat. No. 5,912,253); bioavailability (e.g. U.S. Pat. Nos. 5,221,670; 5,955,457; WO 98/04279); and/or the production of prodrugs (e.g. U.S. Pat. Nos. 6,015,815; 5,432,183) have also been developed. Thus, it is understood in the art that the number of pharmaceutically useful and interesting analogs of rapamycin is very high and difficult to quantify.

A rapamycin analog having similarity to the compound of Formulas I and II is the polyketide disclosed in U.S. Pat. No. 9,382,266. This disclosure provides a stereoisomer of the compound described in U.S. Pat. No. 9,382,266, but U.S. Pat. No. 9,382,266 does not disclose or suggest the novel polyketide provided in the present disclosure, nor does U.S. Pat. No. 9,382,266 provide a composition in which the majority of the polyketide in the composition is the compound of FIG. 1 and/or FIG. 2.

### SUMMARY OF THE DISCLOSURE

In some embodiments, this disclosure provides a polyketide similar to rapamycin that has an unexpected and beneficial pharmaceutical uses. In some embodiments, this disclosure provides compositions comprising the polyketide described in FIG. 1 and/or FIG. 2 and other polyketides. In some embodiments, this disclosure provides novel methods for producing the polyketide of FIG. 1 and/or FIG. 2. In some embodiments, methods of treating a mammal in need thereof comprising administration of the polyketide of FIG. 1 and/or FIG. 2, and/or compositions and/or mixtures comprising the same are provided. Other embodiments are also contemplated as described herein and/or as may be ascertained by those of ordinary skill in the art.

### BRIEF DESCRIPTION OF THE DRAWINGS

FIG. 1: Chemical structure of Formula I.

FIG. 2: Formula II, which is C37-[(1R,2S,4R,5S)-5-hyhroxybicyclo[2.2.1]heptane] rapamycin.

FIG. 3: The rapamycin numbering schema used in this document.

FIG. 4: The chemical structure of commercially available polyketides similar to the polyketide of the present invention, which are rapamycin, temsirolimus, and everolimus.

FIG. 5: The polyketide disclosed in U.S. Pat. No. 9,382,266, C37R-[(1S,2R,4S,5R)-5-hyhroxybicyclo[2.2.1]heptane] rapamycin.

FIG. 6. Dosing of Formula II 2 mg/kg or 10 mg/kg IP (QD×3 days) in Mouse Whole Blood.

FIG. 7. Tumor volume over time. FIG. 7A: Mean Tumor Volume Over Time. FIG. 7B: Median Tumor Volume Over Time; FIG. 7C: Mean Percent Tumor Volume Over Time; FIG. 7D: Median Percent Tumor Volume Over Time.

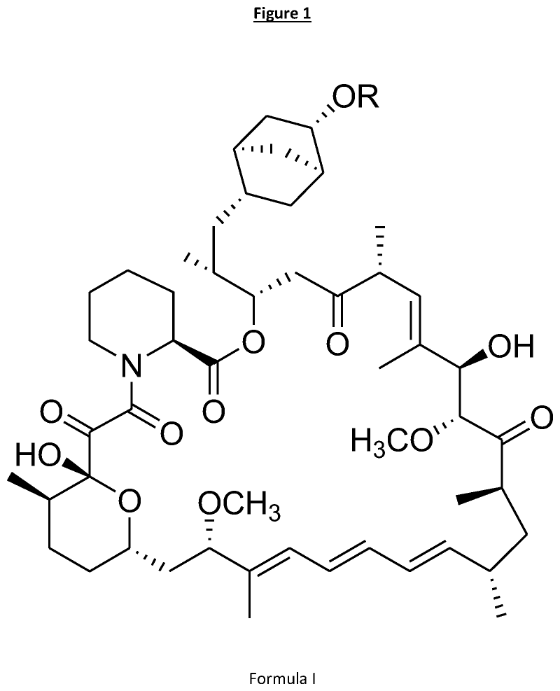

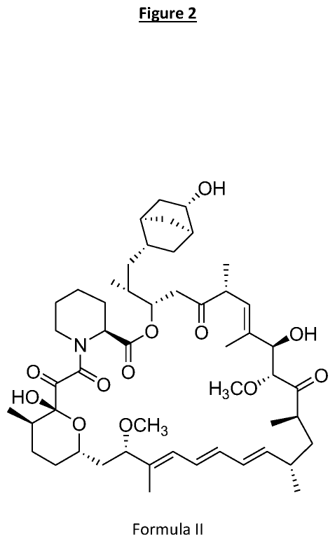

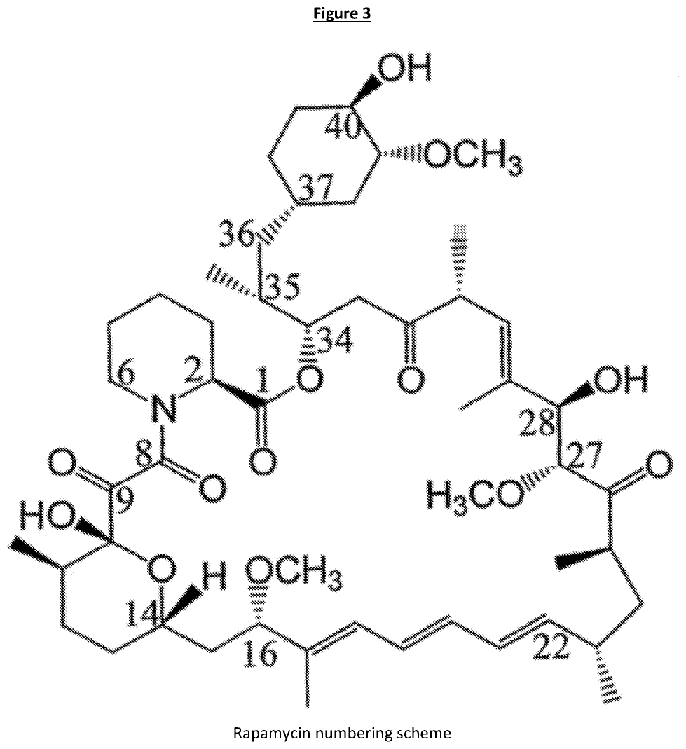

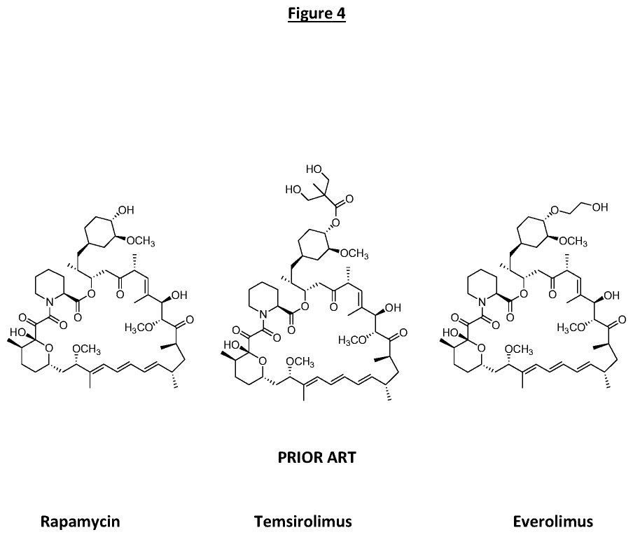

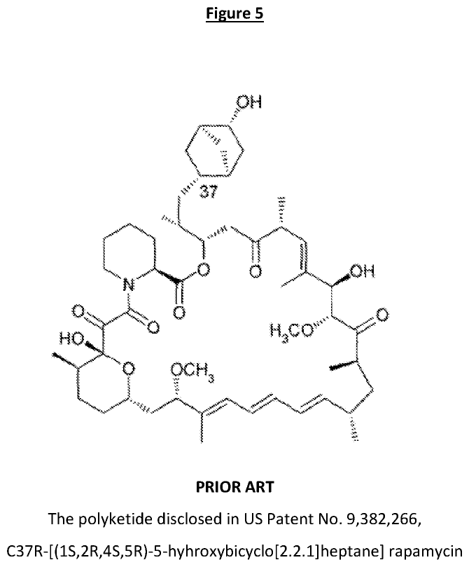

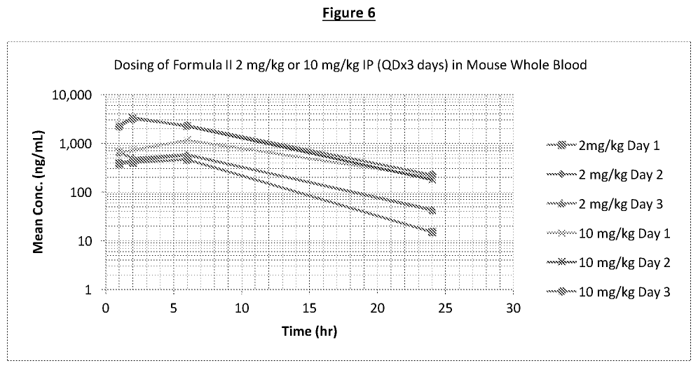

### DETAILED DESCRIPTION

This disclosure provides the polyketides of Formula I and Formula II, which are C37-[(1R,2S,4R,5S)-5-hyhroxybicyclo[2.2.1]heptane] rapamycin and prodrug esters thereof. Compounds of Formula I are prodrugs of the compounds of Formula II which, as described herein, have surprising and unexpectedly beneficial properties for the treatment of mammalian diseases. Formula I is shown below:

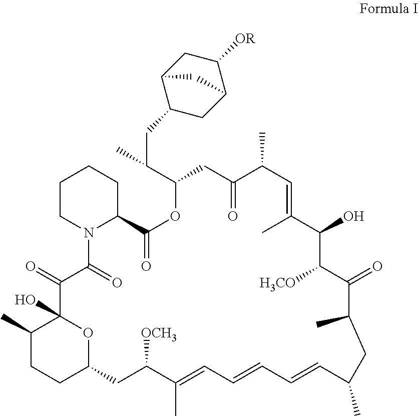

wherein:

R is selected from hydrogen, or —C(O)(CR³R⁴)_{b}(CR⁵R⁶)_{d}(CR⁷R⁸R⁹);

R³ and R⁴ are each, independently, hydrogen, C₁ to C₆ alkyl, C₂ to C₈ alkenyl, C₂ to C₈ alkynyl, trihalomethyl, or —F;

R⁵ and R⁶ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹;

R⁷ is hydrogen, C1 to C₆ alkyl, C₂ to C₈ alkenyl, C₂ to C₈ alkynyl, —(CR³R⁴)_{f}OR¹⁰, —CF₃, —F, or CO₂R¹¹;

R⁸ and R⁹ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹, or R8 and R9 can be taken together to form X or a cycloalkyl ring of 3-8 carbon atoms that is optionally mono-, di-, or tri-substituted with —(CR³R⁴)_{f}OR¹⁰;

R¹⁰ is hydrogen, C₁ to C₆ alkyl, C₂ to C₈ alkenyl, C₂ to C₈ alkynyl, tri-(C₁ to C₆ alkyl)silyl, tri-(C₁ to C₆ alkyl)silylethyl, triphenylmethyl, benzyl, C₂ to C₈ alkoxymethyl, tri-(C₁ to C₆ alkyl)silylethoxymethyl, chloroethyl, or tetrahydropyranyl;

R¹¹ is hydrogen, C1 to C₆ alkyl, C₂ to C₈ alkenyl, C₂ to C₈ alkynyl, or a C₇ to C₁₀ phenylakyl;

X is 5-(2,2-di-(C₁ to C₆ alkyl)[1,3]dioxanyl, 5-(2,2-di-(C₃ to C₈ cycloalkyl)[1,3]dioxanyl, 4-(2,2-di-(C₁ to C₆ alkyl)[1,3]dioxanyl, 4-(2,2-di-(C₃ to C₈ cycloalkyl)[1,3]dioxanyl, 4-(2,2-di-(C₁ to C₆ alkyl)[1,3]dioxalanyl, or 4-(2,2-di-(C₃ to C₈ cycloalkyl)[1,3]dioxalanyl;

b is a whole number from 0 to 6;

d is a whole number from 0 to 6; and,

f is a whole number from 0 to 6.

In a preferred embodiment, R contains at least one moiety selected from —(CR³R⁴)_{f}OR¹⁰, X or —(CR³R⁴)_{f}OR¹⁰ substituted C₃ to C₈ cycloalkyl. Pharmaceutically acceptable salts of such compounds are also provided.

The prodrugs of Formula I are convertible upon administration to a suitable mammal to the compound of Formula II. In some embodiments, the area under the curve formed by a plot of the concentration of the moiety of Formula I administered versus time is less than the area under the curve formed by a plot of the concentration of the compound (or compounds) of Formula II versus time. In some embodiments, the prodrug of formula II is at least 10-fold, and preferably at least 100-fold less pharmaceutically active than a compound of Formula II. In some embodiments, at least 10%, and preferably at least 50%, and more preferably at least 85% of the compound of Formula I is converted to the compound of Formula II during the time following administration to a mammal of the compound of Formula I that is equivalent to the biological half-life of the administered compound of Formula I. In some of the foregoing embodiments, the compound of Formula I is substantially pharmaceutically inert until conversion into the compound of Formula II. However, in other embodiments, the compound of Formula I is significantly pharmaceutically active prior to conversion into the compound of Formula II.

In a preferred embodiment, the polyketide of Formula I is the polyketide of Formula II:

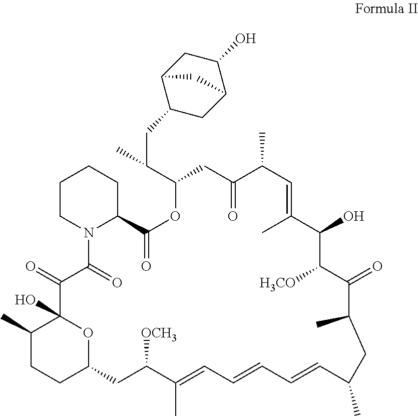 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2C[C@H]3C[C@@H]2C[C@@H]3O)CC(=O)[C@H](C)/C=C(\C)[C@@H](O)[C@@H](OC)C(=O)[C@H](C)C[C@H](C)/C=C/C=C/C=C/1C]

The polyketide disclosed herein, e.g., that of Formulas I or II, despite having structural relatedness to the polyketide disclosed in U.S. Pat. No. 9,382,266, rapamycin, and other analogs of rapamycin, shows a surprising and unexpectedly advantageous pharmacological profile as compared thereto. For instance, the polyketide of Formula II has unexpected advantages for the treatment of certain medical conditions to the polyketide disclosed in U.S. Pat. No. 9,382,266. Other advantages are indicated in Table 1.

TABLE 1
IC₅₀ in PC3 cells

| | TORC1 | TORC2 | TORC1/TORC2 |
|---|---|---|---|
| Test Compound | (nM) | (nM) | Selectivity |
| Formula II, C37-[(1R,2S,4R,5S)-5- | 0.099 | 5 | 50x |
| hyhroxybicyclo[2.2.1]heptane] rapamycin | | | |
| The polyketide disclosed in U.S. 9,382,266: | 0.045 | 11 | 244x |
| C37-[(1S,2R,4S,5R)-5-hyhroxybicyclo[2.2.1]heptane] | | | |
| rapamycin | | | |

The polyketide prodrugs of Formula I, and in particular and preferably the polyketide of Formula II, also have unexpectedly beneficial pharmacokinetics. In particular, the polyketide of Formula II has a high oral bioavailability measured at around 0.47 (% F). This high oral bioavailability is substantially and significantly better than the polyketide disclosed in U.S. Pat. No. 9,382,266 which is about one-half to about one-quarter lower than 0.47 (% F). In an aspect of the present invention, this comparative bioavailability can permit pharmaceutically effective administration of the composition of Formula II with lower toxicity (i.e., an increased therapeutic window). In another aspect of the present invention, this increased bioavailability improves the ability to orally administer the compound of Formula II, relative to the ability to orally administer the polyketide disclosed in U.S. Pat. No. 9,382,266. The benefits of oral administration relative to intravenous and other routes of administration are well understood in the art.

Compositions comprising the polyketide of Formula I or, preferably, Formula II, are optionally, but need not be, pure. The inventive polyketide (of Formula I or, preferably, Formula II) can be present in mixtures in which essentially all of the polyketide in the mixture is the polyketide of Formula I, and preferably Formula II, in which 99.9% by weight of the polyketide in the mixture is the polyketide of Formula I or preferably Formula II, in which 99.5% of the polyketide in the mixture is the polyketide of Formula I or preferably Formula II, in which at least 99% of the polyketide in the mixture is the polyketide of Formula I or preferably Formula II, in which 98% of the polyketide in the mixture is the polyketide of Formula I or preferably Formula II in which at least 95% of the polyketide in the mixture is the polyketide of Formula I or preferably Formula II, in which at least 90% of the polyketide in the mixture is the polyketide of Formula I or preferably Formula II, in which 80% of the polyketide in the mixture is the polyketide of Formula I or preferably Formula II, or in which the 70% of the polyketide in the mixture is the polyketide of Formula I or preferably Formula II.

Furthermore, in each of the foregoing aspects of the present invention the compound of Formula I, and preferably Formula II, is optionally provided as a salt, a solvate, or an ester of the compound of Formula I, and preferably Formula II. Pharmaceutically acceptable salts of the polyketide of the invention include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmitic, malonic, hydroxymaleic, phenylacetic, glutamic, benzoic, salicylic, fumaric, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, optionally can be useful in the preparation of salts useful as intermediates in obtaining the compounds of the invention and their pharmaceutically acceptable salts. More specific examples of suitable basic salts include sodium, lithium, potassium, magnesium, aluminum, calcium, zinc, N,N′-dibenzylethylenediamine, chloroprocaine, choline, diethanolamine, ethylenediamine, N-methylglucamine and procaine salts. In an aspect of the present invention, pharmaceutically acceptable salts of the polyketide of Formula I are combined together with one or more pharmaceutically acceptable excipients, diluents, or carriers.

Similarly, the polyketides of the present invention, and pharmaceutically acceptable salts thereof, optionally can be solvates, including alcoholic solvates and hydrates.

The inventive polyketide (of Formula I or, preferably, Formula II) can be provided in a pure form for example in a crystalline or powdered form or diluted in at least one pharmaceutically acceptable buffer, carrier, or excipient. Pharmaceutically acceptable buffers, carriers and excipients in the context of the present invention preferably do not adversely interact with the polyketide of the present invention, provide for stable formulations for suitable time periods, and are not unduly deleterious to most recipients thereof.

In some embodiments, solutions or suspensions of the inventive polyketide (of Formula I or, preferably, Formula II) also contain excipients such as, e.g., N,N-dimethylacetamide, dispersants e.g. polysorbate 80, surfactants, and solubilizers, e.g. polyethylene glycol, Phosal 50 PG (which consists of phosphatidylcholine, soya-fatty acids, ethanol, mono/diglycerides, propylene glycol and ascorbyl palmitate).

The compositions of the present invention (preferably a compound of Formula I or Formula II, most preferably Formula II) can be administered via any suitable route or means including, but not limited to, parenterally, orally, topically (including buccal, sublingual, or transdermally), via a medical device such as a stent, by inhalation, or via injection (e.g., subcutaneously, intramuscularly, or intravenously). The treatment optionally consists of a single dose, but preferably in many embodiments is a multiplicity of administrations over time. The skilled artisan will recognize that the optimal quantity and spacing of individual dosages of a compound of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

In some embodiments, the compound of Formula I, preferably a compound of Formula II, is administered as the sole active pharmaceutical agent. Thus, in some embodiments, a compound of Formula I, preferably the compound of Formula II, is administered to a mammal such as a human being to prevent and/or treat a disease as a pharmaceutical composition that optionally contains one or more pharmaceutical excipients, but no other active agent(s). In some embodiments, a compound of Formula I, preferably the compound of Formula II, is administered to a mammal such as a human being to prevent and/or treat a disease as a pharmaceutical composition comprising at least one other active agent, and optionally also contains one or more pharmaceutical excipients. In some embodiments, a compound of Formula I, preferably the compound of Formula II, is administered to a mammal such as a human being to prevent and/or treat a disease as a pharmaceutical composition optionally comprising at least one other active agent, and optionally also containing one or more pharmaceutical excipients, with at least one other composition also comprising at least one other active agent and optionally also containing one or more pharmaceutical excipients (e.g., two compositions, one containing at least a compound of Formula I, preferably the compound of Formula II, and the other composition comprising at least one other active agent). Multiple compositions, each comprising one or more active agents (one of such compositions comprising a compound of Formula I, preferably the compound of Formula II), may also be administered to prevent and/or treat disease. Such active agents and/or compositions may be administered simultaneously or sequentially, or some combination thereof, and may be administered at the same or different sites on the mammal, or through the same or different routes of administration.

Active agents that may be administered to a mammal in order to prevent and/or treat disease along with a compound of Formula I, preferably the compound of Formula II, include but are not limited to one or more chemotherapeutic agents, anti-cancer agents, radiation therapy, immune modulators, such as, for instance, and without limitation, one or more of any of: an anti-cancer agent reduces or minimizes any undesired side-effects associated with certain types of cancer treatment (e.g., fatigue, anemia, appetite changes, bleeding problems, diarrhea, constipation, hair loss, nausea, vomiting, pain, peripheral neuropathy, swelling, skin and nail changes, urinary and bladder changes, trouble swallowing, etc.), alkylating agents (e.g., nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, sodium estramustine phosphate, triethylenemelamine, carmustine, lomustine, streptozocin, pipobroman, etoglucid, carboplatin, cisplatin, miboplatin, nedaplatin, oxaliplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin, and the like), antimetabolites (e.g., mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emitefur, and the like), aminopterine, leucovorin calcium, tabloid, butocine, folinate calcium, levofolinate calcium, cladribine, emitefur, fludarabine, gemcitabine, hydroxycarbamide, pentostatin, piritrexim, idoxuridine, mitoguazone, thiazophrine, ambamustine and the like), anticancer antibiotics (e.g., actinomycin-D, actinomycin-C, mitomycin-C, chromomycin-A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, and the like), plant-derived anticancer agents (e.g., etoposide, etoposide phosphate, vinblastine sulfate, vincristine sulfate, vindesine sulfate, teniposide, paclitaxel, docetaxel, vinorelbine, and the like), immunotherapeutic agents (e.g., antibodies (e.g, anti-PD1 antibodies, PD-L1 antibodies, anti-CTLA4 antibodies, anti-CD20 antibodies, anti-CD25 antibodies, HER2 antibody (e.g., trastuzumab), imatinib mesylate, ZD1839 or EGFR antibody (e.g., cetuximab), antibody to VEGF (e.g., bevacizumab), VEGFR antibody, VEGFR inhibitor, and EGFR inhibitor (e.g., erlotinib)), picibanil, krestin, sizofuran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, and the like), methotrexate, doxorubicin, 5-fluorouracil, vincristine, vinblastine, pamidronate disodium, anastrozole, exemestane, cyclophosphamide, epirubicin, toremifene, letrozole, trastuzumab, megestrol, tamoxifen, paclitaxel, docetaxel, capecitabine, goserelin acetate, a glycolytic inhibitor; one or more hormonal therapeutic agents (fosfestrol, diethylstylbestrol, chlorotrianisene, medroxyprogesterone acetate, megestrol acetate, chlormadinone acetate, cyproterone acetate, danazol, dienogest, asoprisnil, allylestrenol, gestrinone, nomegestrol, Tadenan, mepartricin, raloxifene, ormeloxifene, levormeloxifene, anti-estrogens (e.g., tamoxifen citrate, toremifene citrate, and the like), ER down-regulator (e.g., fulvestrant and the like), human menopausal gonadotrophin, follicle stimulating hormone, pill preparations, mepitiostane, testrolactone, aminoglutethimide, LH-RH agonists (e.g., goserelin acetate, buserelin, leuprorelin, and the like), droloxifene, epitiostanol, ethinylestradiol sulfonate, aromatase inhibitors (e.g., fadrozole hydrochloride, anastrozole, retrozole, exemestane, vorozole, formestane, and the like), anti-androgens (e.g., flutamide, bicartamide, nilutamide, and the like), 5α-reductase inhibitors (e.g., finasteride, dutasteride, epristeride, and the like), adrenocorticohormone drugs (e.g., dexamethasone, prednisolone, betamethasone, triamcinolone, and the like); one or more androgen synthesis inhibitors (e.g., abiraterone, and the like); one or more retinoids and/or drugs that retard retinoid metabolism (e.g., liarozole, and the like), etc. and LH-RH agonists (e.g., goserelin acetate, buserelin, leuprorelin)); L-asparaginase, aceglatone, procarbazine hydrochloride, protoporphyrin-cobalt complex salt, mercuric hematoporphyrin-sodium; topoisomerase I inhibitors (e.g., irinotecan, topotecan, and the like), topoisomerase II inhibitors (e.g., sobuzoxane, and the like), differentiation inducers (e.g., retinoid, vitamin D, and the like), α-blockers (e.g., tamsulosin hydrochloride, naftopidil, urapidil, alfuzosin, terazosin, prazosin, silodosin, and the like), serine/threonine kinase inhibitors, endothelin receptor antagonists (e.g., atrasentan, and the like), proteasome inhibitor (e.g., bortezomib, and the like), Hsp 90 inhibitors (e.g., 17-AAG, and the like), spironolactone, minoxidil, 11α-hydroxyprogesterone, bone resorption inhibiting/metastasis suppressing agents (e.g., zoledronic acid, alendronic acid, pamidronic acid, etidronic acid, ibandronic acid, clodronic acid), angiogenesis inhibitors (e.g., nintedanib (BIBF 1120, Vargatef), bevacizumab (Avastin), everolimus (Afinitor), temsirolimus (Torisel), lenalidomide (Revlimid), pazopanib (Votrient), ramucirumab (Cyramza), sorafenib (Nexavar), sunitinib (Sutent), thalidomide (Thalomid), vandetanib (Caprelsa), cediranib (Recentin), axitinib (Inlyta), motesanib, vatalanib, dovitinib, brivanib, linifanib, tivozanib, lenvatinib, regorafenib (Stivarga), foretinib, telatinib, cabozantinib (Cometriq), nilotinib (Tasigna), tandutinib, imatinib (Gleevec), BMS-690514, quizartinib (AC220), orantinib, olaratumab, erlotinib (Tarceva), gefitinib (Iressa), afatinib (Gilotrif), lapatinib (Tykerb), varlitinib, AEE-788, trastuzumab (Herceptin), cetuximab (Erbitux), panitumumab (Vectibix), nimotuzumab, pertuzumab (Omnitarg), ertumaxomab, or zalutumumab. In some embodiments, the angiogenesis inhibitor is nintedanib (BIBF 1120), everolimus (Afinitor), temsirolimus (Torisel), pazopanib (Votrient), axitinib (Inlyta), bevacizumab (Avastin), sorafenib (Nexavar), sunitinib (Sutent), thalidomide (Thalomid), dovitinib, regorafenib (Stivarga), or imatinib (Gleevec)), and the like, and/or combinations and/or mixtures thereof, optionally along with any other active agents described herein or that may be otherwise available to those of skill in the art. A compound of Formula I or Formula II, most preferably Formula II, may also be administered in conjunction with any one or more of surgery, radiotherapy, gene therapy, thermotherapy, cryotherapy, laser cauterization, and the like, and/or any combinations thereof, optionally along with any of the active agents described herein or that may be otherwise available to those of skill in the art.

Tablets containing the inventive polyketide (preferably a compound of Formula I or Formula II, most preferably Formula II) optionally contain excipients such as microcrystalline cellulose, lactose (e.g. lactose monohydrate or lactose anyhydrous), sodium citrate, calcium carbonate, dibasic calcium phosphate and glycine, butylated hydroxytoluene (E321), crospovidone, hypromellose, disintegrants such as starch (preferably corn, potato or tapioca starch), sodium starch glycollate, croscarmellose sodium, and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, and talc are optionally included.

Solid compositions of a similar type can also be employed as fillers in gelatin capsules. Preferred excipients in this regard include lactose, starch, a cellulose, milk sugar or high molecular weight polyethylene glycols. For aqueous suspensions and/or elixirs, the compounds of the invention can be combined with various sweetening or flavoring agents, coloring matter or dyes, with emulsifying and/or suspending agents and with diluents such as water, ethanol, propylene glycol and glycerin, and combinations thereof.

A tablet can be made by compression or molding, optionally with one or more accessory ingredients. Compressed tablets can be prepared by compressing in a suitable machine the active ingredient in a free-flowing form such as a powder or granules, optionally mixed with a binder (e.g. povidone, gelatin, hydroxypropylmethyl cellulose), lubricant, inert diluent, preservative, disintegrant (e.g. sodium starch glycolate, cross-linked povidone, cross-linked sodium carboxymethyl cellulose), surface-active or dispersing agent. Molded tablets can be made by molding in a suitable machine a mixture of the powdered compound moistened with an inert liquid diluent. The tablets can optionally be coated or scored and can be formulated so as to provide slow or controlled release of the active ingredient therein using, for example, hydroxypropylmethylcellulose in varying proportions to provide desired release profile.

For convenience, the formulations are optionally presented in unit dosage form and can be prepared by any of the methods well known in the art of pharmacy. Such methods include the step of bringing into association the active ingredient (compound of the invention) with the carrier which constitutes one or more accessory ingredients. In general, the formulations are prepared by uniformly and intimately bringing into association the active ingredient with liquid carriers or finely divided solid carriers or both, and then, if necessary, shaping the product.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollient in creams or ointments and ethanol or oeyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

For parenteral administration, fluid unit dosage forms are prepared utilizing the active ingredient and a sterile vehicle, for example but without limitation water, alcohols, polyols, glycerine and vegetable oils, water being preferred. The active ingredient, depending on the vehicle and concentration used, can be either suspended or dissolved in the vehicle. In preparing solutions, the active ingredient can be dissolved in water for injection and filter sterilized before filling into a suitable vial or ampoule and sealing.

Advantageously, agents such as local anesthetics, preservatives and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use.

Parenteral suspensions are prepared in substantially the same manner as solutions, except that the active ingredient is suspended in the vehicle instead of being dissolved and sterilization cannot be accomplished by filtration. The active ingredient can be sterilized by exposure to ethylene oxide before suspending in the sterile vehicle. Advantageously, a surfactant or wetting agent is included in the composition to facilitate uniform distribution of the active ingredient.

The compound of Formula I and/or Formula II may also be administered using medical devices known in the art. For example, in one embodiment, a pharmaceutical composition described herein can be administered with a needleless hypodermic injection device, such as the devices disclosed in U.S. Pat. Nos. 5,399,163; 5,383,851; 5,312,335; 5,064,413; 4,941,880; 4,790,824; or U.S. Pat. No. 4,596,556. Useful examples of well-known implants and modules include but are not limited to U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. In a specific embodiment, the polyketides (e.g., of Formula I or II) and compositions comprising the same may be administered using a drug-eluting stent, for example, such as one corresponding to those described in WO 01/87263 and related publications or those described by Perin (Perin, E C, 2005). Many other such implants, delivery systems, and modules are known to those skilled in the art.

The polyketides and compositions described herein comprising a polyketide of Formula I, and/or preferably Formula II, can be administered to treat, prevent, or mitigate a disease or medical condition in a mammal in need thereof. In a preferred embodiment, the mammal is a human. Any appropriate medical condition of the mammal can be treated by administering a pharmaceutically-appropriate quantity of the formulation of Formula I, and preferably of Formula II, to a mammal in need thereof. An ordinarily skill artisan can readily select the route of administration of the inventive polyketide (of Formula I or, preferably, Formula II) as well as the quantity following routine studies, guidelines and procedures. The dosage to be administered of a compound of the invention will vary according to the particular compound, the disease involved, the subject, and the nature and severity of the disease and the physical condition of the subject, and the selected route of administration. The appropriate dosage can be readily determined by a person skilled in the art. For example, without limitation, a dose of about 0.1 mg up to 100 mg daily, and optionally about 0.1 to 15 mg daily (or a higher dose given less frequently) may be contemplated.

The compositions may contain any suitable combination of the inventive polyketide (of Formula I or, preferably, Formula II) and other components. In some preferred embodiments, the compositions of the invention contain from 0.1 weight % to 70 weight % of the inventive polyketide (of Formula I or, preferably, Formula II), preferably from 5-60 weight %, more preferably from 10 to 30 weight %, of the inventive polyketide (of Formula I or, preferably, Formula II), depending on the method of administration and other factors.

While not desiring to be bound by theory, it is believed that the adverse side effects associated with the administration of rapamycin-related polyketides are caused more by mTORC2 inhibition than mTORC1 inhibition. Thus, the skilled artisan may prefer polyketides that are selective for mTORC1 for conditions in which cellular escape mechanisms are not of particularly high concern. On the other hand, cancerous cells are well-known to exhibit rapid genomic plasticity that can result in the development of drug resistant cancer in the mammal treated. For these diseases, it will often be desirable to administer an mTOR inhibiting polyketide that inhibits mTORC1 and mTORC2 in a more balanced fashion. The polyketides of Formula I, specifically including the polyketides of Formula II, unexpectedly and advantageously inhibit mTORC1 and mTORC2 in a more balanced way than does the polyketide disclosed in U.S. Pat. No. 9,382,266. While not desiring to be bound by theory, the present invention provides a method of treating a mammal in need thereof comprising administering a polyketide disclosed herein (e.g., of Formula I and/or Formula II) to the mammal, wherein the condition to be treated is selected from cancer and other proliferative dysplasias, fungal infections, and systemic lupus erythematosus. In an aspect of the present invention, the cancerous condition is lymphangioleiomyomatosis, a leukemia, renal cell carcinoma, ovarian cancer, pancreatic cancer or a lymphoma. Other aspects are also described herein.

The polyketide of Formula I and/or Formula II described herein can be produced as a direct fermentation product, by feeding a starter acid of formula (III).

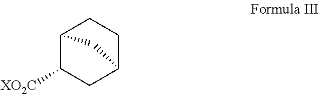

Suitable conditions for such a process are described in WO 2004/007709 (US 2005/0272132 A1) and WO 2006/016167 (US 2009/0253732 A1), the contents of which are incorporated by reference in their entirety. Specifically, a mutant strain of the rapamycin producing organism, Streptomyces hygroscopicus, that lacks the rapK gene and is called S. hygroscopicus ArapK (BIOT-4010; See, Example 1 of U.S. Pat. No. 9,382,266, the methods and materials of which are herein incorporated by reference) was generated. Other suitable production strains include S. hygroscopicus MG2-10 (pLL178), a derivative of S. hygroscopicus NRRL5491. The generation of S. hygroscopicus MG2-10 is described in example 2 of WO 2004/007709 (US 2005/0272132 A1), and to generate a suitable production strain, this should be complemented with rapIJMNOQL, using an expression plasmid such pLL178 (as described in example 7 of WO 2006/016167 (US 2009/0253732 A1)). Fermentation of BIOT-4010, or a similar strain, such as S. hygroscopicus MG2-10 (pLL178) (WO 2004/007709 (US 2005/0272132 A1), WO 2006/016167 (US 2009/0253732 A1)) in a suitable medium, such as (but not limited to) MD6, at a suitable temperature, such as 26° C., with addition of exo-(1R,2S,4R,5S)-5-hydroxybicyclo[2.2.1]heptane-2-carboxylate, typically at 24 hours is then sufficient for the production of the compound of the invention. Peak titers are observed between 3 and 8 days from inoculation. The acid form of compound of formula (II) is exo-(1R,2S,4R,5S)-5-hydroxybicyclo[2.2.1]heptane-2-carboxylic acid.

Rapamycin producing strains include Streptomyces hygroscopicus, Actinoplates sp. N902-109 (See, Nishida et al (1995)) and Streptomyces sp. A 91-261402 (See, WO 94/18207). Other rapamycin producing strains are mentioned in WO 95/06649. The contents of WO 94/18207 and WO 95/06649 are incorporated in the present patent document by reference in their entirety.

The compound of the invention can be purified, for example, from other fermentation products, including but not limited to other polyketides, by any suitable conventional separation techniques, such as but not limited to, flash chromatography, preparative HPLC, and/or crystallization.

Accordingly, in one aspect, the present invention provides a process for preparing a compound of the invention in substantially pure form comprises the steps of (i) feeding a starter acid of formula (III):

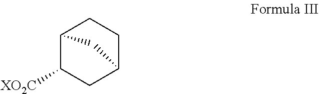

to a rapamycin producing strain that has been genetically altered either to remove or inactivate the rapK gene, or in other embodiments, to remove or inactivate a gene encoding a chorismatase with function equivalent to that of the rapK gene product RapK (See, Andexer et al., 2011); and (ii) isolating and purifying the compound of the invention.

Compounds of Formula I can be prepared by acylation of the compound of Formula II using protected hydroxyl and polypro acids, alkoxy or polyalkoxy carboxylic acids that have been activated, followed by removal of the alcohol protecting groups if so desired. Several procedures for carboxylate activation are known in the art, but the preferred methods utilize carbodiimides, mixed anhydrides, or acid chlorides. For example, an appropriately substituted carboxylic acid can be activated as a mixed anhydride, with an acylating group such as 2,4,6-trichlorobenzoyl anhydride. Treatment of Formula II with the mixed anhydride under mildly basic condition provides the desired compounds. Alternatively, the acylation reaction can be accomplished with 1-(3-dimethylaminopropyl)-3-ethylcarbodiimide hydrochloride and dimethylaminopyridine.

Vulnerable hydroxyls of Formula II can be protected during the synthesis of Formula I through routine addition of a suitable protecting group such as tert-butyl dimethylsilyl protecting group, which at a later stage can be removed under mildly acidic conditions such as in a solution of acetic acid/water/THF. Deprotection is further described in U.S. Pat. No. 5,118,678, which is hereby incorporated by reference. Alternative synthetic methods are provided by the analogy to the methods in U.S. Pat. No. 5,120,842, which is hereby incorporated by reference.

The compounds of Formula I and Formula II can be purified by any suitable separation technology including, but not limited to, preparative-scale chromatography.

Thus, in some embodiments, this disclosure provides a compound of Formula I as described above, and/or a pharmaceutically acceptable salt, solvate, ester, or mixture thereof. In some embodiments, this disclosure provides a composition comprising such a compound, pharmaceutically acceptable salt, solvate, ester, or mixture and, optionally, at least one pharmaceutically acceptable carrier. In some embodiments, this disclosure provides a prodrug of Formula II, wherein the prodrug is a polyketide of Formula I, as well as pharmaceutically acceptable salts, solvates, and hydrates of the compound of Formula I. In some embodiments, this disclosure provides a compound of Formula II as well as pharmaceutically acceptable salts, solvates, esters, or mixtures thereof, and/or compositions comprising the same (e.g., pharmaceutical compositions comprising a pharmaceutically acceptable carrier). In some embodiments, this disclosure provides a composition comprising about 70% or more, about 80% or more, about 90% or more (i.e., “substantially pure”), about 95% or more, or about 99% or more of a compound selected from the group consisting of the compound of Formula II, a pharmaceutically acceptable salt thereof, a solvate thereof, an ester thereof of the compound of formula I, and/or mixtures of the foregoing. In some embodiments, the composition contains an essentially pure mixture, wherein an essentially pure mixture may contain trace amounts or pharmaceutically insignificant amounts of other polyketides, of a compound selected from the group consisting of the compound of Formula I and preferably Formula II, pharmaceutically acceptable salts, solvates, and esters of the compound of Formula I, and preferably Formula (II), and mixtures of the foregoing. In some embodiments, this disclosure provides a pharmaceutical composition comprising a pharmaceutically acceptable carrier, diluent or excipient and a polyketide of Formula I and preferably Formula II, wherein the pharmaceutically acceptable salt, solvate, and/or hydrate of the compound of Formula I and preferably Formula II comprises at least about 70%, at least about 80%, at least about 90%, at least about 95%, or at least about 98% of the polyketide component of the pharmaceutical composition. In some embodiments of such compositions, that polyketide compound is essentially the only polyketide in the pharmaceutical composition. In some embodiments of such compositions, the prodrug of Formula I may be substituted for the polyketide of Formula II. In some embodiments, the solvate, if present, is a hydrate.

In some embodiments, method of inhibiting the proliferation of a cell, the method comprising contacting said cell with an antiproliferative amount of a compound of Formula II, pharmaceutically acceptable salt thereof, solvate thereof, ester thereof, or mixture thereof and/or comprising Formula II; and/or a composition comprising Formula II. In some embodiments, the cell is human cell such as, preferably a human cancer cell (such as but not limited to, e.g., adenocarcinoma, bladder cancer, blood cancer, bone cancer, brain cancer, solid tumor, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and/or transitional cell carcinoma). In some embodiments, the method is an in vitro method or an in vivo method. In some embodiments, the antiproliferative effect of the compound of Formula II is determined using the cell count EC₅₀, the IC₅₀, and/or GI50. In some exemplary embodiments, the EC₅₀ is about 1 E-03 (0.001) micromolar or less, or between 1 E-03 (0.001) and 7.17E-05 (0.0000717) micromolar, and or at least about 10% that of rapamycin. In some exemplary embodiments, the cell count IC₅₀ is about 1 E-01 (0.1) micromolar or less, or between about 1 E-01 (0.1) and about 2.97E-04 (0.000297) micromolar, and/or at least about 10% that of rapamycin. In some exemplary embodiments, the cell count GI₅₀ is 1 E-02 (0.01) micromolar, between 1 E-02 (0.01) and about 8.72E-04 micromolar, and/or at least about 10% that of rapamycin.

In some embodiments, this disclosure also provides methods for preventing and/or treating cancer, the method comprising administering to said mammal (e.g., a human being) an effective amount (e.g., a therapeutically effective amount) of the compound, pharmaceutically acceptable salt thereof, a solvate thereof, an ester thereof of the compound of Formula I or Formula II, preferably Formula II, and/or a composition and/or mixture comprising the same. In some embodiments, the method of treating a mammal in need thereof comprises administering to said mammal an effective amount of a compound of Formula II, pharmaceutically acceptable salt thereof, solvate thereof, ester thereof, or mixture thereof and/or comprising the compound of Formula II; and/or a composition comprising Formula II (e.g., a therapeutically effective amount) thereto. In some embodiments, the mammal has a disease selected from the group consisting of cancer (such as but not limited to, e.g., a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma), systemic lupus erythematosus, pulmonary inflammation, and/or is in need of prevention of organ transplant rejection or host-versus-graft disease. In some embodiments, the compound of Formula I and/or Formula II are administered as the sole active pharmaceutical agent(s); or the compound(s) of Formula I and/or Formula II are administered in combination with one or more of a chemotherapeutic agent, anti-cancer agent, or immune modulator; and/or radiation therapy and/or surgery. In some embodiments, the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately. In some embodiments, the compound(s) of Formula I and/or II are provided as a bead, tablet, capsule, solution, or suspension. In some embodiments, this disclosure provides the use of a compound of Formula I and/or Formula I in the preparation of a medicament for the prevention and/or treatment of cancer. In some embodiments, the method comprises administration of the compound of Formula II to a mammal at about 2 mg/kg to provide an approximate mean concentration of 350-700 ng/mL (e.g., 383-651 ng/mL) in the whole blood of the mammal for up to six hours following administration. In some embodiments, the method comprises administration of the compound of Formula II to a mammal at about 2 mg/kg to provide an approximate mean concentration of 15-50 ng/mL (e.g., 15-43.7 ng/mL) in the whole blood of the mammal at about 24 hours following administration. In some embodiments, the method comprises administration of the compound of Formula II to a mammal at about 10 mg/kg to provides= an approximate mean concentration of from 600-3500 ng/mL (e.g., 657-3323 ng/mL) in the whole blood of the mammal for up to six hours following administration. In some embodiments, the method comprises administration of the compound of Formula II to a mammal at about 10 mg/kg to provides an approximate mean concentration of from 20-150 ng/mL (e.g., 21-138 ng/mL) in the whole blood of the mammal at about 24 hours following administration. In some embodiments, the method comprises administering the compound of Formula II to a mammal having a solid tumor and multiple administrations to the mammal are performed, and resulting in a reduction in the volume of the solid tumor (e.g., at least about any of 20%, 25%, 30%, 40%, 50%, or 60%). In some such embodiments, a significant reduction in tumor volume results from administration of Formula II for about eight consecutive days. In some embodiments, a significant reduction as measured by tumor percent is exhibited following administration of the compound of Formula II to the mammal for about four consecutive days. In some embodiments, such as those using an animal model (e.g., a mouse), the compound of Formula II is administered to the mammal for 30 days and results mean differential tumor percent as compared to an untreated mammal of about 0.7291 with a 95% confidence interval of about 0.3481 to about 1.11 with an adjusted P value of 0.0001 as determined by Dunnett's multiple comparison's test. In some embodiments, this disclosure provides a kit for preventing and/or treating cancer comprising at least one therapeutically effective dose of the compound of Formula I and/or Formula II, and instructions for preventing and/or treating cancer using the same.

Any mode of administration may be utilized. In some embodiments, the compound, composition and/or mixture is administered by application to an implantable medical device (e.g., a stent).

In some embodiments, this disclosure also provides processes for preparing a compound of Formula (I), or a pharmaceutically acceptable salt thereof, which processes comprise a feeding starter exo-(1R,2S,4R,5S)-5-hydroxybicyclo[2.2.1]heptane-2-carboxylate of formula (III):

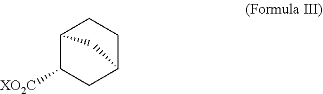

where X═H, alkyl, sodium or potassium, to a rapamycin-producing strain that has been genetically altered to remove or inactivate the rapK gene or a homologue thereof.

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto. As used herein, a subject or a host or a mammal is meant to be an individual. The subject can include mammals such as domesticated animals, such as cats and dogs, livestock (e.g., cattle, horses, pigs, sheep, and goats), laboratory animals (e.g., mice, rabbits, rats, guinea pigs) and birds. A mammal may also be a primate or a human. Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different compounds or molecules or may not include a combination such that the description includes both the combination and the absence of the combination (i.e., individual members of the combination). The term “combined” or “in combination” or “in conjunction” may refer to a physical combination of agents that are administered together or the use of two or more agents in a regimen (e.g., administered separately, physically and/or in time) for treating, preventing and/or ameliorating a particular disease. Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed. Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps. All references referred to in this application, including patent and patent applications, are incorporated herein by reference into this disclosure in their entirety.

Certain embodiments are further described in the following examples. These embodiments are provided as examples only and are not intended to limit the scope of the claims in any way

### EXAMPLES

### Example 1

This example demonstrates that the polyketide of the present invention inhibits mTORC1 and mTORC2 less selectively than the to the polyketide disclosed in U.S. Pat. No. 9,382,266. The data from this example is disclosed in Table 1.

A. Cell Culture

PC3 cells were maintained in F12K media supplemented with 10% FBS, 1% Penicillin/Streptomycin, 2 mM L-glutamine and cultured at 37° C. under an atmosphere of 95% air and 5% CO₂. Cells were treated with 100 nM of Example (I) for 24 hours and harvested in RIPA buffer (300 nM NaCl, 1.0% NP-40, 0.5% sodium deoxycholate, 0.1% SDS, 50 mM Tris (pH 8.0), protease inhibitor cocktail (Roche), phosphatase inhibitor 2, 3 (Sigma). Protein concentrations were determined using a detergent compatible, Lowry-like protein assay (DC protein assay, Biorad).

B. Western Blot Analysis

Equal amounts of protein were resolved by SDS-PAGE and transferred to nitrocellulose membrane using the Invitrogen Nu-Page system. The membranes were blocked for 1 hr in 5% milk and incubated overnight in the following appropriate antibodies: anti-rpS6 (Cell Signaling #2217), anti-phospho-rpS6 (Cell Signaling #2211), anti-phospho Akt (S473) (Cell Signaling #4691). The following day, blots were washed 3 times in TBST, incubated for 2 hr with secondary antibodies (donkey anti rabbit hrp conjugated), and finally washed an additional 3 times in TBST. ECL Prime (Amersham) was used to detect the proteins of interest and ImageJ was used to quantify blots. The data are reported in Table 1 herein. This example demonstrates that the polyketide of Formula II inhibits mTORC1 and mTORC2 less selectively than the to the polyketide disclosed in U.S. Pat. No. 9,382,266.

### Example 2

This example compares the inhibition of proliferation the indicated cell lines, compared with that observed for staurosporin and rapamycin, using the OncoPanel™ cell proliferation assay which measures the proliferation response of cancer cell lines to drug treatments through high-content fluorescence imaging or bioluminescence.

Cells were grown in RPMI 1640, 10% FBS, 2 mM L-alanyl-L-glutamine, 1 mM Na pyruvate, or a special medium. Cells were seeded into 384-well plates and incubated in a humidified atmosphere of 5% CO₂ at 37° C. Compounds were added the day following cell seeding. At the same time, a time zero untreated cell plate was generated. After a 3-day incubation period, cells were fixed and stained to allow fluorescence imaging of nuclei. Compounds (1 mM stock solutions) were serially diluted in half-log steps from the highest test concentration (1 micromol), and assayed over 10 concentrations with a maximum assay concentration of 0.1% DMSO. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software.

Cell proliferation was measured by the fluorescence intensity of an incorporated nuclear dye. The output is referred to as the relative cell count, where the measured nuclear intensity is transformed to percent of control (POC) using the following formula:

$$POC = \frac{{I}_{x}}{{I}_{0}} \times 100$$

Where Iₓ is the nuclear intensity at concentration x, and I₀ is the average nuclear intensity of the untreated vehicle wells.

Cellular response parameters were calculated using nonlinear regression to a sigmoidal single-site dose response model:

$$y = A + \frac{B - A}{1 + {( C / x )}^{D}}$$

Where y is a response measured at concentration x, A and B are the lower and upper limits of the response, C is the concentration at the response midpoint (EC₅₀), and D is the Hill Slope (Ref.1).

Time zero non-treated plates were used to determine the number of doublings during the assay period, using the formula:

$$Doublings = {log}_{2} ( \frac{N}{{N}_{T 0}} )$$

Where N is the cell number in untreated wells at the assay end point and N_{T0} is the cell number at the time of compound addition.

Cell count IC₅₀ is the test compound concentration at 50% of maximal possible response. EC₅₀ is the test compound concentration at the curve inflection point or half the effective response (parameter C of the fitted curve solution). GI₅₀ is the concentration needed to reduce the observed growth by half (midway between the curve maximum and the time zero value). “Cell Count Activity Area” is an estimate of the integrated area above the curve (Barretina, et al. The Cancer Cell Line Encyclopedia enables predictive modelling of anticancer drug sensitivity. Nature 483: 603-607). Cell Count Activity Area Values Range from 0-10, where a Value of Zero Indicates no inhibition of proliferation at all concentrations, and a value of 10 indicates complete inhibition of proliferation at all concentrations. In rare instances, values <0 or >10 may be observed. In these instances, values <0 should be considered as equivalent to 0, whereas values >10 should be considered equivalent to 10. Curve-fitting, calculations, and report generation was performed using a custom data reduction engine and MathIQ based software (AIM). Vehicle background effects on the tested cell lines are summarized in Table 2:

TABLE 2

| | | Relative cell count (POC) | | | |
|---|---|---|---|---|---|
| Plate # | Cell line | Mean | Std Dev | CV | Doublings |
| 6 | SCaBER | 100.00 | 11.86 | 0.12 | 3.26 |
| 6 | SHP-77 | 100.00 | 4.45 | 0.04 | 1.68 |
| 6 | SK-N-AS | 100.00 | 4.84 | 0.05 | 1.96 |
| 6 | SNU-C2B | 100.00 | 7.16 | 0.07 | 1.40 |
| 6 | SK-NEP-1 | 100.00 | 4.59 | 0.05 | 1.49 |
| 6 | RKOE6 | 100.00 | 5.06 | 0.05 | 3.08 |
| 6 | OVCAR3 | 100.00 | 6.42 | 0.06 | 1.38 |
| 6 | NCI-H510A | 100.00 | 16.66 | 0.17 | 1.25 |
| 6 | RL95-2 | 100.00 | 3.30 | 0.03 | 1.30 |
| 6 | PC-3 | 100.00 | 6.83 | 0.07 | 2.67 |
| 6 | U-138MG | 100.00 | 5.16 | 0.05 | 1.31 |
| 6 | U2OS | 100.00 | 5.76 | 0.06 | 2.74 |
| 6 | TF-1 | 100.00 | 5.05 | 0.05 | 1.69 |
| 6 | U-118 MG | 100.00 | 7.09 | 0.07 | 1.12 |
| 6 | U-87 MG | 100.00 | 4.88 | 0.05 | 1.54 |
| 6 | ST486 | 100.00 | 6.54 | 0.07 | 1.29 |
| 6 | SK-UT-1 | 100.00 | 6.08 | 0.06 | 2.77 |
| 6 | SNU-5 | 100.00 | 1.50 | 0.01 | 1.40 |
| 6 | T24 | 100.00 | 6.03 | 0.06 | 3.44 |
| 6 | SW579 | 100.00 | 11.79 | 0.12 | 2.60 |
| 6 | A101D | 100.00 | 4.54 | 0.05 | 1.61 |
| 6 | A388 | 100.00 | 6.62 | 0.07 | 2.36 |
| 6 | Hs 852.T | 100.00 | 7.60 | 0.08 | 1.49 |
| 6 | AGS | 100.00 | 10.76 | 0.11 | 3.47 |
| 6 | A431 | 100.00 | 7.87 | 0.08 | 3.50 |
| 6 | NCIH446 | 100.00 | 10.59 | 0.11 | 1.19 |
| 6 | SW684 | 100.00 | 9.07 | 0.09 | 0.92 |
| 6 | G-402 | 100.00 | 17.97 | 0.18 | 2.46 |
| 6 | 786-O | 100.00 | 4.33 | 0.04 | 3.16 |
| 6 | Hs 729 | 100.00 | 5.22 | 0.05 | 0.85 |
| 6 | CaOV3 | 100.00 | 4.06 | 0.04 | 1.79 |
| 6 | DMS114 | 100.00 | 3.42 | 0.03 | 0.72 |
| 6 | CML-T1 | 100.00 | 2.11 | 0.02 | 2.77 |
| 6 | MES-SA | 100.00 | 3.83 | 0.04 | 3.26 |
| 6 | SK-MEL-28 | 100.00 | 3.83 | 0.04 | 1.63 |
| 6 | AsPC-1 | 100.00 | 4.21 | 0.04 | 1.20 |
| 6 | CAMA-1 | 100.00 | 6.44 | 0.06 | 1.44 |
| 6 | CRO-AP2 | 100.00 | 2.97 | 0.03 | 3.37 |
| 6 | CHP-212 | 100.00 | 2.81 | 0.03 | 1.21 |
| 6 | CEM-C1 | 100.00 | 4.09 | 0.04 | 3.06 |

The results of these assays performed using staurosporine, rapamycin, and the compound of Formula II are shown in Table 3.

TABLE 3

| | | Cell Count EC50 | Cell Count IC50 | Cell Count GI50 | Cell Count |
|---|---|---|---|---|---|
| Compound | Cell Line | (microM) | (microM) | (microM) | Activity Area |
| Staurosporine | G-402 | 9.42E−04 | 1.23E−03 | 6.64E−04 | 5.93 |
| Rapamycin | G-402 | 1.11E−04 | 3.34E−04 | 1.82E−04 | 4.68 |
| Formula II | G-402 | 1.13E−03 | 2.87E−03 | 1.51E−03 | 4.75 |
| Staurosporine | NCIH446 | 3.26E−02 | 5.02E−02 | 1.68E−02 | 2.81 |
| Rapamycin | NCIH446 | >1.00E+00 | >1.00E+00 | 4.44E−02 | 1.69 |
| Formula II | NCIH446 | >1.00E+00 | >1.00E+00 | >1.00E+00 | 1.23 |
| Staurosporine | SW684 | 1.15E−03 | >1.00E+00 | 9.50E−04 | 3.15 |
| Rapamycin | SW684 | 2.23E−03 | >1.00E+00 | 1.97E−03 | 2.81 |
| Formula II | SW684 | 4.57E−03 | 3.70E−01 | 2.19E−03 | 2.97 |
| Staurosporine | Hs 729 | 2.06E−03 | 7.04E−03 | 8.98E−04 | 4.06 |
| Rapamycin | Hs 729 | >1.00E+00 | >1.00E+00 | >1.00E+00 | 0.55 |
| Formula II | Hs 729 | >1.00E+00 | >1.00E+00 | 1.01E−01 | 0.91 |
| Staurosporine | 786-O | 3.58E−03 | 4.33E−03 | 3.41E−03 | 4.87 |
| Rapamycin | 786-O | 2.27E−04 | 3.18E−03 | 1.13E−03 | 4.17 |
| Formula II | 786-O | 1.59E−03 | 1.62E−02 | 7.10E−03 | 4.01 |
| Staurosporine | Hs 852.T | 9.30E−04 | 5.85E−03 | 1.35E−03 | 3.16 |
| Rapamycin | Hs 852.T | >1.00E+00 | >1.00E+00 | >1.00E+00 | 1.43 |
| Formula II | Hs 852.T | >1.00E+00 | >1.00E+00 | >1.00E+00 | 0.95 |
| Staurosporine | A101D | 7.02E−03 | 8.74E−03 | 2.65E−03 | 4.56 |
| Rapamycin | A101D | 1.84E−04 | >1.00E+00 | 7.21E−04 | 2.92 |
| Formula II | A101D | 3.66E−04 | 5.62E−02 | 1.36E−03 | 3.79 |
| Staurosporine | A388 | 9.17E−04 | 1.62E−03 | 1.03E−03 | 4.99 |
| Rapamycin | A388 | >1.00E+00 | >1.00E+00 | >1.00E+00 | 2.25 |
| Formula II | A388 | 8.24E−03 | >1.00E+00 | >1.00E+00 | 1.60 |
| Staurosporine | A431 | 9.53E−04 | 1.19E−03 | 1.03E−03 | 5.64 |
| Rapamycin | A431 | >1.00E+00 | >1.00E+00 | >1.00E+00 | 0.28 |
| Formula II | A431 | 1.21E−02 | >1.00E+00 | >1.00E+00 | 1.43 |
| Staurosporine | AGS | 1.16E−03 | 1.21E−03 | 9.26E−04 | 6.19 |
| Rapamycin | AGS | 3.87E−05 | 3.14E−04 | 1.65E−04 | 5.67 |
| Formula II | AGS | 1.33E−04 | 2.97E−04 | 2.45E−04 | 5.29 |
| Staurosporine | CRO-AP2 | 1.36E−02 | 1.43E−02 | 1.32E−02 | 3.99 |
| Rapamycin | CRO-AP2 | 1.19E−04 | 1.87E−04 | 1.55E−04 | 6.22 |
| Formula II | CRO-AP2 | 1.07E−03 | 2.21E−03 | 1.65E−03 | 4.89 |
| Staurosporine | AsPC-1 | 1.99E−03 | 3.93E−03 | 2.05E−03 | 3.28 |
| Rapamycin | AsPC-1 | >1.00E+00 | >1.00E+00 | >1.00E+00 | 1.48 |
| Formula II | AsPC-1 | 8.56E−03 | >1.00E+00 | 2.46E−01 | 1.68 |
| Staurosporine | CAMA-1 | 2.02E−02 | 9.90E−02 | 1.20E−02 | 2.99 |
| Rapamycin | CAMA-1 | 1.94E−04 | 1.19E−03 | 2.25E−04 | 4.07 |
| Formula II | CAMA-1 | 4.91E−04 | 4.34E−03 | 6.47E−04 | 4.22 |
| Staurosporine | CEM-C1 | 7.60E−03 | 9.20E−03 | 7.27E−03 | 4.28 |
| Rapamycin | CEM-C1 | 2.65E−04 | >1.00E+00 | 4.36E−03 | 3.61 |
| Formula II | CEM-C1 | 1.80E−03 | 2.65E−01 | 3.25E−02 | 3.13 |
| Staurosporine | CHP-212 | 4.19E−03 | 4.78E−03 | 2.59E−03 | 4.80 |
| Rapamycin | CHP-212 | 3.76E−05 | >1.00E+00 | 7.07E−05 | 4.13 |
| Formula II | CHP-212 | 2.67E−04 | 3.40E−02 | 3.49E−04 | 3.87 |
| Staurosporine | CML-T1 | 7.06E−03 | 1.02E−02 | 6.96E−03 | 4.13 |
| Rapamycin | CML-T1 | 1.89E−04 | 1.93E−03 | 6.94E−04 | 4.56 |
| Formula II | CML-T1 | 8.21E−04 | 1.25E−02 | 3.64E−03 | 3.68 |
| Staurosporine | CaOV3 | 2.44E−04 | 5.39E−04 | 2.73E−04 | 5.31 |
| Rapamycin | CaOV3 | >1.00E+00 | >1.00E+00 | >1.00E+00 | 2.40 |
| Formula II | CaOV3 | 1.59E−03 | >1.00E+00 | 4.77E−02 | 2.53 |
| Staurosporine | DMS114 | 2.10E−02 | 5.68E−02 | 3.35E−03 | 3.04 |
| Rapamycin | DMS114 | 7.47E−05 | >1.00E+00 | 4.78E−05 | 4.14 |
| Formula II | DMS114 | 1.34E−03 | 3.31E−02 | 6.11E−04 | 3.40 |
| Staurosporine | SK-MEL-28 | 5.39E−02 | 6.43E−02 | 3.21E−02 | 2.81 |
| Rapamycin | SK-MEL-28 | 6.54E−04 | >1.00E+00 | 9.87E−02 | 2.50 |
| Formula II | SK-MEL-28 | 4.71E−03 | >1.00E+00 | 3.82E−02 | 2.68 |
| Staurosporine | MES-SA | 1.25E−02 | 1.42E−02 | 1.30E−02 | 2.80 |
| Rapamycin | MES-SA | 1.74E−04 | 4.15E−04 | 2.94E−04 | 5.39 |
| Formula II | MES-SA | 1.17E−03 | 2.74E−03 | 1.96E−03 | 4.81 |
| Staurosporine | NCI-H510A | 3.98E−02 | >1.00E+00 | 5.65E−02 | 1.68 |
| Rapamycin | NCI-H510A | >1.00E+00 | >1.00E+00 | >1.00E+00 | 1.63 |
| Formula II | NCI-H510A | 7.17E−05 | >1.00E+00 | >1.00E+00 | 2.57 |
| Staurosporine | RKOE6 | 1.68E−02 | 1.79E−02 | 1.32E−02 | 3.96 |
| Rapamycin | RKOE6 | 8.35E−05 | 1.06E−03 | 3.56E−04 | 4.61 |
| Formula II | RKOE6 | 1.13E−03 | 1.42E−02 | 5.57E−03 | 3.66 |
| Staurosporine | OVCAR3 | 7.87E−03 | 1.73E−02 | 6.11E−03 | 3.41 |
| Rapamycin | OVCAR3 | 1.53E−03 | >1.00E+00 | 1.24E−01 | 2.08 |
| Formula II | OVCAR3 | 4.26E−02 | >1.00E+00 | 3.01E−01 | 1.09 |
| Staurosporine | PC-3 | 9.03E−04 | 1.31E−03 | 1.07E−03 | 4.67 |
| Rapamycin | PC-3 | 2.30E−04 | >1.00E+00 | 2.63E−03 | 3.57 |
| Formula II | PC-3 | 6.37E−04 | 1.84E−02 | 3.99E−03 | 3.80 |
| Staurosporine | RL95-2 | 1.86E−02 | 2.01E−02 | 6.83E−03 | 3.87 |
| Rapamycin | RL95-2 | 7.05E−05 | >1.00E+00 | 1.30E−04 | 4.24 |
| Formula II | RL95-2 | 6.45E−04 | >1.00E+00 | 1.18E−03 | 3.36 |
| Staurosporine | SK-N-AS | 1.70E−03 | 3.86E−03 | 1.27E−03 | 4.83 |
| Rapamycin | SK-N-AS | 2.31E−04 | >1.00E+00 | 4.32E−04 | 3.58 |
| Formula II | SK-N-AS | 1.41E−03 | 4.22E−01 | 4.82E−03 | 3.12 |
| Staurosporine | SCaBER | 4.22E−04 | 5.45E−04 | 4.74E−04 | 6.03 |
| Rapamycin | SCaBER | 3.77E−05 | 3.21E−04 | 1.58E−04 | 5.25 |
| Formula II | SCaBER | 2.50E−04 | 1.59E−03 | 8.72E−04 | 4.82 |
| Staurosporine | SHP-77 | 2.81E−02 | 3.08E−02 | 2.42E−02 | 3.16 |
| Rapamycin | SHP-77 | >1.00E+00 | >1.00E+00 | >1.00E+00 | 1.13 |
| Formula II | SHP-77 | >1.00E+00 | >1.00E+00 | >1.00E+00 | 0.76 |
| Staurosporine | SK-NEP-1 | 4.82E−02 | 5.45E−02 | 3.65E−02 | 2.78 |
| Rapamycin | SK-NEP-1 | 2.93E−04 | 1.47E−03 | 3.61E−04 | 4.22 |
| Formula II | SK-NEP-1 | 2.48E−03 | 1.81E−02 | 3.27E−03 | 3.36 |
| Staurosporine | SNU-C2B | 1.58E−02 | 2.47E−02 | 4.97E−03 | 3.80 |
| Rapamycin | SNU-C2B | >1.00E+00 | >1.00E+00 | >1.00E+00 | 1.99 |
| Formula II | SNU-C2B | 1.79E−04 | >1.00E+00 | 3.16E−03 | 2.73 |
| Staurosporine | SNU-5 | 6.44E−03 | 2.28E−01 | 1.03E−02 | 2.58 |
| Rapamycin | SNU-5 | 8.85E−04 | >1.00E+00 | 2.40E−02 | 2.23 |
| Formula II | SNU-5 | 5.57E−03 | >1.00E+00 | 1.68E−01 | 1.85 |
| Staurosporine | ST486 | 4.22E−03 | 4.61E−03 | 2.83E−03 | 4.89 |
| Rapamycin | ST486 | 3.54E−05 | >1.00E+00 | 5.24E−05 | 3.99 |
| Formula II | ST486 | 5.81E−04 | >1.00E+00 | 1.17E−03 | 3.05 |
| Staurosporine | SK-UT-1 | 2.18E−02 | 2.86E−02 | 1.95E−02 | 3.46 |
| Rapamycin | SK-UT-1 | 2.00E−04 | >1.00E+00 | >1.00E+00 | 3.23 |
| Formula II | SK-UT-1 | 1.51E−03 | 3.82E−02 | 1.13E−02 | 3.48 |
| Staurosporine | SW579 | 5.20E−04 | 6.69E−04 | 5.06E−04 | 6.09 |
| Rapamycin | SW579 | 1.36E−05 | >1.00E+00 | 5.39E−05 | 4.77 |
| Formula II | SW579 | 2.04E−04 | 4.52E−03 | 1.06E−03 | 4.51 |
| Staurosporine | T24 | 8.01E−04 | 9.53E−04 | 8.29E−04 | 5.96 |
| Rapamycin | T24 | 7.83E−05 | 2.10E−03 | 4.74E−04 | 4.39 |
| Formula II | T24 | 4.71E−04 | 6.80E−03 | 3.09E−03 | 4.16 |
| Staurosporine | TF-1 | 1.08E−02 | 1.19E−02 | 1.09E−02 | 3.31 |
| Rapamycin | TF-1 | >1.00E+00 | >1.00E+00 | >1.00E+00 | 1.52 |
| Formula II | TF-1 | >1.00E+00 | >1.00E+00 | >1.00E+00 | 1.08 |
| Staurosporine | U-138MG | 8.57E−04 | 5.44E−03 | 1.05E−03 | 3.72 |
| Rapamycin | U-138MG | 3.63E−05 | >1.00E+00 | 4.36E−04 | 3.14 |
| Formula II | U-138MG | 1.44E−03 | >1.00E+00 | 2.30E−02 | 2.59 |
| Staurosporine | U2OS | 2.72E−03 | 3.40E−03 | 2.63E−03 | 4.91 |
| Rapamycin | U2OS | 6.50E−04 | >1.00E+00 | 2.54E−03 | 3.04 |
| Formula II | U2OS | 5.40E−03 | 3.24E−01 | 3.50E−02 | 2.61 |
| Staurosporine | U-87 MG | 7.52E−04 | 2.46E−03 | 8.36E−04 | 3.25 |
| Rapamycin | U-87 MG | 1.04E−04 | >1.00E+00 | 3.84E−04 | 3.79 |
| Formula II | U-87 MG | 9.96E−04 | 3.41E−02 | 2.17E−03 | 3.76 |
| Staurosporine | U-118 MG | 1.00E−03 | >1.00E+00 | 1.93E−03 | 3.04 |
| Rapamycin | U-118 MG | 7.92E−05 | >1.00E+00 | 1.24E−04 | 2.87 |
| Formula II | U-118 MG | 2.86E−04 | >1.00E+00 | 3.20E−04 | 3.11 |

A further detailed analysis of the effect of the compound of Formula II on proliferation of each cell line is provided in the Table 4 below:

TABLE 4
G-402
Human renal leiomyoblastoma

| | Relative cell count (%) | |
|---|---|---|
| Concentration (microM) | Mean | StdDev |
| 3.18E − 05 | 97.8 | 7.8 |
| 1.01E − 04 | 100.0 | 14.2 |
| 3.18E − 04 | 86.8 | 0.9 |
| 1.00E − 03 | 63.7 | 8.5 |
| 3.17E − 03 | 52.6 | 11.4 |
| 1.00E − 02 | 40.5 | 8.5 |
| 3.17E − 02 | 36.6 | 6.8 |
| 1.00E − 01 | 28.4 | 2.1 |
| 3.16E − 01 | 27.5 | 2.8 |
| 1.00E + 00 | 25.8 | 10.1 |

NCIH446
Small cell lung carcinoma

| | Relative cell count (%) | |
|---|---|---|
| Concentration (microM) | Mean | StdDev |
| 3.18E − 05 | 94.2 | 11.2 |
| 1.01E − 04 | 102.5 | 9.9 |
| 3.18E − 04 | 87.3 | 6.7 |
| 1.00E − 03 | 93.7 | 18.2 |
| 3.17E − 03 | 85.8 | 6.3 |
| 1.00E − 02 | 83.2 | 14.5 |
| 3.17E − 02 | 83.3 | 17.6 |
| 1.00E − 01 | 83.6 | 11.2 |
| 3.16E − 01 | 74.9 | 5.7 |
| 1.00E + 00 | 80.9 | 6.0 |

SW684
Human fibrosarcoma

| | Relative cell count (%) | |
|---|---|---|
| Concentration (microM) | Mean | StdDev |
| 3.18E − 05 | 99.5 | 4.4 |
| 1.01E − 04 | 94.1 | 3.5 |
| 3.18E − 04 | 86.4 | 2.8 |
| 1.00E − 03 | 85.1 | 4.0 |
| 3.17E − 03 | 71.8 | 4.3 |
| 1.00E − 02 | 71.4 | 8.5 |
| 3.17E − 02 | 63.3 | 4.8 |
| 1.00E − 01 | 55.6 | 2.7 |
| 3.16E − 01 | 52.4 | 0.9 |
| 1.00E + 00 | 49.0 | 1.4 |

Hs 729
Human rhabdomyosarcoma

| | Relative cell count (%) | |
|---|---|---|
| Concentration (microM) | Mean | StdDev |
| 3.18E − 05 | 93.2 | 5.5 |
| 1.01E − 04 | 105.0 | 8.3 |
| 3.18E − 04 | 93.3 | 2.0 |
| 1.00E − 03 | 97.1 | 4.4 |
| 3.17E − 03 | 98.0 | 2.7 |
| 1.00E − 02 | 91.1 | 5.3 |
| 3.17E − 02 | 79.3 | 4.0 |
| 1.00E − 01 | 81.0 | 5.2 |
| 3.16E − 01 | 74.8 | 3.9 |
| 1.00E + 00 | 73.3 | 4.9 |

786-0
Human renal cell adenocarcinoma

| | Relative cell count (%) | |
|---|---|---|
| Concentration (microM) | Mean | StdDev |
| 3.18E − 05 | 96.8 | 4.7 |
| 1.01E − 04 | 92.0 | 2.6 |
| 3.18E − 04 | 80.1 | 4.3 |
| 1.00E − 03 | 78.6 | 2.4 |
| 3.17E − 03 | 65.0 | 6.6 |
| 1.00E − 02 | 55.7 | 3.1 |
| 3.17E − 02 | 48.2 | 2.9 |
| 1.00E − 01 | 45.5 | 3.7 |
| 3.16E − 01 | 43.3 | 0.8 |
| 1.00E + 00 | 36.7 | 2.1 |

Hs 852.T
Human melanoma

| | Relative cell count (%) | |
|---|---|---|
| Concentration (microM) | Mean | StdDev |
| 3.18E − 05 | 95.0 | 6.6 |
| 1.01E − 04 | 98.7 | 5.9 |
| 3.18E − 04 | 92.8 | 2.7 |
| 1.00E − 03 | 92.9 | 2.2 |
| 3.17E − 03 | 90.6 | 7.4 |
| 1.00E − 02 | 91.2 | 7.4 |
| 3.17E − 02 | 82.3 | 7.1 |
| 1.00E − 01 | 80.1 | 7.8 |
| 3.16E − 01 | 73.2 | 4.0 |
| 1.00E + 00 | 72.0 | 1.7 |

A101D
Human melanoma

| | Relative cell count (%) | |
|---|---|---|
| Concentration (microM) | Mean | StdDev |
| 3.18E − 05 | 100.8 | 18.1 |
| 1.01E − 04 | 96.5 | 5.3 |
| 3.18E − 04 | 82.7 | 5.5 |
| 1.00E − 03 | 75.1 | 0.5 |
| 3.17E − 03 | 71.5 | 6.6 |
| 1.00E − 02 | 62.0 | 1.5 |
| 3.17E − 02 | 56.2 | 6.2 |
| 1.00E − 01 | 54.3 | 4.2 |
| 3.16E − 01 | 59.6 | 3.3 |
| 1.00E + 00 | 52.5 | 3.5 |

A388
Human epidermoid carcinoma

| | Relative cell count (%) | |
|---|---|---|
| Concentration (microM) | Mean | StdDev |
| 3.18E − 05 | 86.5 | 9.2 |
| 1.01E − 04 | 95.5 | 1.1 |
| 3.18E − 04 | 87.7 | 4.8 |
| 1.00E − 03 | 80.8 | 3.0 |
| 3.17E − 03 | 79.0 | 1.1 |
| 1.00E − 02 | 79.8 | 2.8 |
| 3.17E − 02 | 66.7 | 6.8 |
| 1.00E − 01 | 65.6 | 1.4 |
| 3.16E − 01 | 62.3 | 6.8 |
| 1.00E + 00 | 62.7 | 1.0 |

A431
Human epidermoid carcinoma

| | Relative cell count (%) | |
|---|---|---|
| Concentration (microM) | Mean | StdDev |
| 3.18E − 05 | 100.9 | 8.6 |
| 1.01E − 04 | 103.1 | 8.4 |
| 3.18E − 04 | 102.0 | 4.2 |
| 1.00E − 03 | 94.6 | 6.3 |
| 3.17E − 03 | 96.4 | 2.1 |
| 1.00E − 02 | 85.0 | 4.7 |
| 3.17E − 02 | 81.8 | 1.2 |
| 1.00E − 01 | 80.3 | 7.1 |
| 3.16E − 01 | 75.3 | 4.9 |
| 1.00E + 00 | 71.4 | 6.7 |

AGS
Human gastric adenocarcinoma

| | Relative cell count (%) | |
|---|---|---|
| Concentration (microM) | Mean | StdDev |
| 3.18E − 05 | 124.5 | 11.7 |
| 1.01E − 04 | 87.1 | 9.5 |
| 3.18E − 04 | 63.8 | 2.0 |
| 1.00E − 03 | 48.7 | 8.0 |
| 3.17E − 03 | 51.4 | 8.8 |
| 1.00E − 02 | 41.0 | 2.7 |
| 3.17E − 02 | 42.8 | 3.3 |
| 1.00E − 01 | 44.3 | 2.3 |
| 3.16E − 01 | 40.7 | 3.0 |
| 1.00E + 00 | 41.7 | 1.1 |

CRO-AP2
Human B cell lymphoma

| | Relative cell count (%) | |
|---|---|---|
| Concentration (microM) | Mean | StdDev |
| 3.18E − 05 | 98.0 | 1.4 |
| 1.01E − 04 | 100.6 | 3.0 |
| 3.18E − 04 | 81.8 | 4.7 |
| 1.00E − 03 | 65.3 | 0.7 |
| 3.17E − 03 | 47.3 | 1.9 |
| 1.00E − 02 | 34.9 | 1.6 |
| 3.17E − 02 | 27.0 | 1.9 |
| 1.00E − 01 | 27.5 | 0.7 |
| 3.16E − 01 | 24.7 | 0.1 |
| 1.00E + 00 | 24.8 | 1.6 |

AsPC-1
Human adenocarcinoma

| | Relative cell count (%) | |
|---|---|---|
| Concentration (microM) | Mean | StdDev |
| 3.18E − 05 | 104.0 | 2.6 |
| 1.01E − 04 | 105.4 | 8.3 |
| 3.18E − 04 | 97.5 | 5.9 |
| 1.00E − 03 | 90.9 | 1.3 |
| 3.17E − 03 | 90.9 | 3.4 |
| 1.00E − 02 | 89.3 | 2.9 |
| 3.17E − 02 | 81.7 | 2.9 |
| 1.00E − 01 | 74.4 | 6.3 |
| 3.16E − 01 | 77.3 | 2.7 |
| 1.00E + 00 | 70.3 | 1.9 |

CAMA-1
Human breast adenocarcinoma

| | Relative cell count (%) | |
|---|---|---|
| Concentration (microM) | Mean | StdDev |
| 3.18E − 05 | 96.5 | 1.0 |
| 1.01E − 04 | 98.0 | 2.0 |
| 3.18E − 04 | 79.7 | 8.8 |
| 1.00E − 03 | 64.5 | 3.0 |
| 3.17E − 03 | 55.9 | 4.7 |
| 1.00E − 02 | 54.6 | 2.3 |
| 3.17E − 02 | 45.1 | 2.7 |
| 1.00E − 01 | 45.4 | 0.9 |
| 3.16E − 01 | 43.0 | 2.2 |
| 1.00E + 00 | 42.0 | 1.4 |

CEM-C1
Human acute lymphoblastic leukemia

| | Relative cell count (%) | |
|---|---|---|
| Concentration (microM) | Mean | StdDev |
| 3.18E − 05 | 100.6 | 5.0 |
| 1.01E − 04 | 98.1 | 3.8 |
| 3.18E − 04 | 92.1 | 6.1 |
| 1.00E − 03 | 82.0 | 4.6 |
| 3.17E − 03 | 73.0 | 2.6 |
| 1.00E − 02 | 66.8 | 0.9 |
| 3.17E − 02 | 58.8 | 3.3 |
| 1.00E − 01 | 56.2 | 2.3 |
| 3.16E − 01 | 52.4 | 2.8 |
| 1.00E + 00 | 51.5 | 0.1 |

CHP-212
Human neuroblastoma

| | Relative cell count (%) | |
|---|---|---|
| Concentration (microM) | Mean | StdDev |
| 3.18E − 05 | 93.4 | 1.3 |
| 1.01E − 04 | 82.6 | 2.5 |
| 3.18E − 04 | 68.8 | 1.4 |
| 1.00E − 03 | 59.5 | 5.3 |
| 3.17E − 03 | 59.2 | 4.0 |
| 1.00E − 02 | 57.6 | 6.4 |
| 3.17E − 02 | 54.5 | 6.5 |
| 1.00E − 01 | 47.0 | 1.9 |
| 3.16E − 01 | 46.9 | 2.5 |
| 1.00E + 00 | 43.7 | 3.5 |

CML-T1
Human chronic myelogenous leukemia

| | Relative cell count (%) | |
|---|---|---|
| Concentration (microM) | Mean | StdDev |
| 3.18E − 05 | 96.6 | 1.9 |
| 1.01E − 04 | 96.1 | 1.1 |
| 3.18E − 04 | 78.8 | 2.3 |
| 1.00E − 03 | 67.7 | 3.0 |
| 3.17E − 03 | 59.5 | 5.6 |
| 1.00E − 02 | 54.5 | 1.2 |
| 3.17E − 02 | 46.2 | 0.9 |
| 1.00E − 01 | 46.4 | 2.0 |
| 3.16E − 01 | 42.4 | 3.0 |
| 1.00E + 00 | 44.0 | 2.3 |

CaOV3
Human ovary adenocarcinoma

| | Relative cell count (%) | |
|---|---|---|
| Concentration (microM) | Mean | StdDev |
| 3.18E − 05 | 97.5 | 1.1 |
| 1.01E − 04 | 98.2 | 9.3 |
| 3.18E − 04 | 76.6 | 2.0 |
| 1.00E − 03 | 76.4 | 1.7 |
| 3.17E − 03 | 78.3 | 2.0 |
| 1.00E − 02 | 73.9 | 4.0 |
| 3.17E − 02 | 64.6 | 2.4 |
| 1.00E − 01 | 63.1 | 4.3 |
| 3.16E − 01 | 60.5 | 3.8 |
| 1.00E + 00 | 57.7 | 2.6 |

DMS114
Human lung carcinoma

| | Relative cell count (%) | |
|---|---|---|
| Concentration (microM) | Mean | StdDev |
| 3.18E − 05 | 99.7 | 5.5 |
| 1.01E − 04 | 102.9 | 14.1 |
| 3.18E − 04 | 83.4 | 2.3 |
| 1.00E − 03 | 74.7 | 1.7 |
| 3.17E − 03 | 63.9 | 3.0 |
| 1.00E − 02 | 62.2 | 0.8 |
| 3.17E − 02 | 52.5 | 0.6 |
| 1.00E − 01 | 50.1 | 1.1 |
| 3.16E − 01 | 46.7 | 1.9 |
| 1.00E + 00 | 43.1 | 0.6 |

SK-MEL-28
Human malignant melanoma

| | Relative cell count (%) | |
|---|---|---|
| Concentration (microM) | Mean | StdDev |
| 3.18E − 05 | 102.8 | 10.2 |
| 1.01E − 04 | 110.7 | 9.5 |
| 3.18E − 04 | 94.6 | 1.8 |
| 1.00E − 03 | 91.6 | 3.1 |
| 3.17E − 03 | 85.4 | 3.0 |
| 1.00E − 02 | 84.3 | 2.6 |
| 3.17E − 02 | 71.0 | 3.9 |
| 1.00E − 01 | 69.8 | 2.2 |
| 3.16E − 01 | 66.4 | 1.1 |
| 1.00E + 00 | 60.6 | 2.4 |

MES-SA
Human uterine sarcoma

| | Relative cell count (%) | |
|---|---|---|
| Concentration (microM) | Mean | StdDev |
| 3.18E − 05 | 105.0 | 5.0 |
| 1.01E − 04 | 107.7 | 5.8 |
| 3.18E − 04 | 90.9 | 5.0 |
| 1.00E − 03 | 71.1 | 9.0 |
| 3.17E − 03 | 53.2 | 5.7 |
| 1.00E − 02 | 43.5 | 0.4 |
| 3.17E − 02 | 35.6 | 0.9 |
| 1.00E − 01 | 31.4 | 2.0 |
| 3.16E − 01 | 26.5 | 0.8 |
| 1.00E + 00 | 26.6 | 0.7 |

NCI-H510A
Human metastatic small cell lung cancer

| | Relative cell count (%) | |
|---|---|---|
| Concentration (microM) | Mean | StdDev |
| 3.18E − 05 | 129.0 | 2.0 |
| 1.01E − 04 | 102.2 | 7.4 |
| 3.18E − 04 | 101.6 | 3.5 |
| 1.00E − 03 | 92.3 | 2.7 |
| 3.17E − 03 | 95.4 | 6.2 |
| 1.00E − 02 | 99.6 | 3.8 |
| 3.17E − 02 | 90.4 | 2.1 |
| 1.00E − 01 | 84.2 | 4.9 |
| 3.16E − 01 | 88.0 | 6.0 |
| 1.00E + 00 | 91.4 | 6.8 |

RKOE6
Human epithelial carcinoma (colon)

| | Relative cell count (%) | |
|---|---|---|
| Concentration (microM) | Mean | StdDev |
| 3.18E − 05 | 98.6 | 1.5 |
| 1.01E − 04 | 94.2 | 6.6 |
| 3.18E − 04 | 84.4 | 8.8 |
| 1.00E − 03 | 64.1 | 3.9 |
| 3.17E − 03 | 65.3 | 11.1 |
| 1.00E − 02 | 52.2 | 5.1 |
| 3.17E − 02 | 49.1 | 1.9 |
| 1.00E − 01 | 43.1 | 5.8 |
| 3.16E − 01 | 43.1 | 3.7 |
| 1.00E + 00 | 40.1 | 9.5 |

OVCAR3
Human ovary adenocarcinoma

| | Relative cell count (%) | |
|---|---|---|
| Concentration (microM) | Mean | StdDev |
| 3.18E − 05 | 99.9 | 8.8 |
| 1.01E − 04 | 116.6 | 3.0 |
| 3.18E − 04 | 100.4 | 3.7 |
| 1.00E − 03 | 101.9 | 4.6 |
| 3.17E − 03 | 98.5 | 2.6 |
| 1.00E − 02 | 104.2 | 4.7 |
| 3.17E − 02 | 88.4 | 6.4 |
| 1.00E − 01 | 79.0 | 3.8 |
| 3.16E − 01 | 69.1 | 5.3 |
| 1.00E + 00 | 70.8 | 6.4 |

PC-3
Human prostate adenocarcinoma

| | Relative cell count (%) | |
|---|---|---|
| Concentration (microM) | Mean | StdDev |
| 3.18E − 05 | 96.9 | 10.5 |
| 1.01E − 04 | 98.3 | 3.2 |
| 3.18E − 04 | 73.9 | 1.3 |
| 1.00E − 03 | 70.9 | 2.8 |
| 3.17E − 03 | 61.5 | 4.3 |
| 1.00E − 02 | 59.2 | 2.5 |
| 3.17E − 02 | 50.8 | 0.6 |
| 1.00E − 01 | 50.3 | 1.6 |
| 3.16E − 01 | 47.6 | 1.4 |
| 1.00E + 00 | 42.2 | 2.5 |

RL95-2
Human uterus carcinoma

| | Relative cell count (%) | |
|---|---|---|
| Concentration (microM) | Mean | StdDev |
| 3.18E − 05 | 95.1 | 1.7 |
| 1.01E − 04 | 91.3 | 3.2 |
| 3.18E − 04 | 78.8 | 2.0 |
| 1.00E − 03 | 69.2 | 2.4 |
| 3.17E − 03 | 62.6 | 1.8 |
| 1.00E − 02 | 61.8 | 4.3 |
| 3.17E − 02 | 53.8 | 4.0 |
| 1.00E − 01 | 51.9 | 3.3 |
| 3.16E − 01 | 51.5 | 0.9 |
| 1.00E + 00 | 48.1 | 4.0 |

SK-N-AS
Human neuroblastoma

| | Relative cell count (%) | |
|---|---|---|
| Concentration (microM) | Mean | StdDev |
| 3.18E − 05 | 97.7 | 2.1 |
| 1.01E − 04 | 99.5 | 7.6 |
| 3.18E − 04 | 89.7 | 5.4 |
| 1.00E − 03 | 77.8 | 2.0 |
| 3.17E − 03 | 70.3 | 5.0 |
| 1.00E − 02 | 55.3 | 5.1 |
| 3.17E − 02 | 55.1 | 1.8 |
| 1.00E − 01 | 54.0 | 4.2 |
| 3.16E − 01 | 49.5 | 2.4 |
| 1.00E + 00 | 49.7 | 1.7 |

SCaBER
Human squamous cell carcinoma (bladder)

| | Relative cell count (%) | |
|---|---|---|
| Concentration (microM) | Mean | StdDev |
| 3.18E − 05 | 93.1 | 10.6 |
| 1.01E − 04 | 83.1 | 3.6 |
| 3.18E − 04 | 63.5 | 3.2 |
| 1.00E − 03 | 56.1 | 3.3 |
| 3.17E − 03 | 50.7 | 6.9 |
| 1.00E − 02 | 42.2 | 3.4 |
| 3.17E − 02 | 39.5 | 2.9 |
| 1.00E − 01 | 44.5 | 11.6 |
| 3.16E − 01 | 36.3 | 5.1 |
| 1.00E + 00 | 32.7 | 5.9 |

SHP-77
Human small cell lung carcinoma

| | Relative cell count (%) | |
|---|---|---|
| Concentration (microM) | Mean | StdDev |
| 3.18E − 05 | 98.5 | 7.2 |
| 1.01E − 04 | 95.8 | 6.1 |
| 3.18E − 04 | 90.6 | 8.1 |
| 1.00E − 03 | 96.1 | 6.9 |
| 3.17E − 03 | 92.3 | 4.3 |
| 1.00E − 02 | 96.8 | 6.3 |
| 3.17E − 02 | 88.0 | 1.0 |
| 1.00E − 01 | 79.9 | 2.2 |
| 3.16E − 01 | 75.2 | 2.3 |
| 1.00E + 00 | 67.9 | 5.2 |

SK-NEP-1
Human metastatic kidney cancer

| | Relative cell count (%) | |
|---|---|---|
| Concentration (microM) | Mean | StdDev |
| 3.18E − 05 | 99.0 | 8.7 |
| 1.01E − 04 | 106.4 | 4.1 |
| 3.18E − 04 | 94.6 | 5.3 |
| 1.00E − 03 | 86.5 | 5.8 |
| 3.17E − 03 | 67.1 | 6.9 |
| 1.00E − 02 | 59.8 | 5.6 |
| 3.17E − 02 | 49.3 | 0.8 |
| 1.00E − 01 | 45.4 | 2.9 |
| 3.16E − 01 | 44.3 | 3.2 |
| 1.00E + 00 | 41.8 | 1.8 |

SNU-C2B
Human colorectal carcinoma

| | Relative cell count (%) | |
|---|---|---|
| Concentration (microM) | Mean | StdDev |
| 3.18E − 05 | 106.6 | 8.6 |
| 1.01E − 04 | 97.1 | 1.8 |
| 3.18E − 04 | 88.6 | 5.9 |
| 1.00E − 03 | 74.1 | 6.4 |
| 3.17E − 03 | 76.3 | 8.6 |
| 1.00E − 02 | 79.1 | 2.9 |
| 3.17E − 02 | 69.5 | 1.9 |
| 1.00E − 01 | 76.2 | 0.4 |
| 3.16E − 01 | 66.6 | 4.8 |
| 1.00E + 00 | 74.9 | 7.9 |

SNU-5
Human metastatic gastric carcinoma

| | Relative cell count (%) | |
|---|---|---|
| Concentration (microM) | Mean | StdDev |
| 3.18E − 05 | 101.0 | 2.9 |
| 1.01E − 04 | 101.2 | 0.4 |
| 3.18E − 04 | 95.6 | 1.8 |
| 1.00E − 03 | 95.6 | 1.6 |
| 3.17E − 03 | 84.9 | 0.6 |
| 1.00E − 02 | 82.9 | 1.9 |
| 3.17E − 02 | 76.3 | 1.0 |
| 1.00E − 01 | 71.9 | 1.3 |
| 3.16E − 01 | 70.4 | 1.7 |
| 1.00E + 00 | 67.1 | 1.9 |

ST486
Human Burkitt's Lymphoma (B cell)

| | Relative cell count (%) | |
|---|---|---|
| Concentration (microM) | Mean | StdDev |
| 3.18E − 05 | 92.2 | 3.3 |
| 1.01E − 04 | 90.5 | 9.6 |
| 3.18E − 04 | 77.5 | 5.8 |
| 1.00E − 03 | 72.7 | 10.5 |
| 3.17E − 03 | 58.2 | 4.8 |
| 1.00E − 02 | 57.6 | 4.5 |
| 3.17E − 02 | 55.2 | 2.2 |
| 1.00E − 01 | 56.2 | 1.6 |
| 3.16E − 01 | 49.9 | 2.5 |
| 1.00E + 00 | 55.2 | 3.6 |

SK-UT-1
Human uterine tumor

| | Relative cell count (%) | |
|---|---|---|
| Concentration (microM) | Mean | StdDev |
| 3.18E − 05 | 116.9 | 15.0 |
| 1.01E − 04 | 114.4 | 7.7 |
| 3.18E − 04 | 96.2 | 4.5 |
| 1.00E − 03 | 82.8 | 8.1 |
| 3.17E − 03 | 75.4 | 2.0 |
| 1.00E − 02 | 76.1 | 10.3 |
| 3.17E − 02 | 61.4 | 4.9 |
| 1.00E − 01 | 59.0 | 5.0 |
| 3.16E − 01 | 53.3 | 1.9 |
| 1.00E + 00 | 47.0 | 5.3 |

SW579
Human squamous cell carcinoma

| | Relative cell count (%) | |
|---|---|---|
| Concentration (microM) | Mean | StdDev |
| 3.18E − 05 | 86.0 | 8.2 |
| 1.01E − 04 | 82.8 | 10.0 |
| 3.18E − 04 | 66.2 | 3.6 |
| 1.00E − 03 | 64.1 | 5.1 |
| 3.17E − 03 | 50.2 | 2.8 |
| 1.00E − 02 | 51.1 | 3.5 |
| 3.17E − 02 | 49.9 | 3.8 |
| 1.00E − 01 | 43.4 | 9.2 |
| 3.16E − 01 | 40.8 | 4.9 |
| 1.00E + 00 | 43.1 | 4.7 |

T24
Human bladder transitional cell carcinoma

| | Relative cell count (%) | |
|---|---|---|
| Concentration (microM) | Mean | StdDev |
| 3.18E − 05 | 93.2 | 4.6 |
| 1.01E − 04 | 85.7 | 8.5 |
| 3.18E − 04 | 69.8 | 3.0 |
| 1.00E − 03 | 61.8 | 7.7 |
| 3.17E − 03 | 52.6 | 1.6 |
| 1.00E − 02 | 52.8 | 4.7 |
| 3.17E − 02 | 48.5 | 6.7 |
| 1.00E − 01 | 42.2 | 2.7 |
| 3.16E − 01 | 40.8 | 1.1 |
| 1.00E + 00 | 36.6 | 0.3 |

TF-1
Human erythroleukemia (bone marrow)

| | Relative cell count (%) | |
|---|---|---|
| Concentration (microM) | Mean | StdDev |
| 3.18E − 05 | 94.4 | 2.1 |
| 1.01E − 04 | 93.1 | 5.2 |
| 3.18E − 04 | 89.0 | 7.0 |
| 1.00E − 03 | 88.7 | 2.8 |
| 3.17E − 03 | 80.8 | 1.6 |
| 1.00E − 02 | 82.8 | 2.0 |
| 3.17E − 02 | 81.7 | 2.7 |
| 1.00E − 01 | 80.1 | 2.8 |
| 3.16E − 01 | 79.6 | 2.5 |
| 1.00E + 00 | 80.8 | 1.9 |

U-138MG
Human glioblastoma

| | Relative cell count (%) | |
|---|---|---|
| Concentration (microM) | Mean | StdDev |
| 3.18E − 05 | 87.8 | 0.8 |
| 1.01E − 04 | 87.7 | 3.7 |
| 3.18E − 04 | 78.5 | 4.0 |
| 1.00E − 03 | 74.8 | 4.6 |
| 3.17E − 03 | 73.8 | 4.6 |
| 1.00E − 02 | 76.4 | 6.3 |
| 3.17E − 02 | 69.1 | 2.7 |
| 1.00E − 01 | 67.9 | 1.2 |
| 3.16E − 01 | 65.0 | 4.1 |
| 1.00E + 00 | 60.4 | 1.4 |

U2OS
Human osteosarcoma

| | Relative cell count (%) | |
|---|---|---|
| Concentration (microM) | Mean | StdDev |
| 3.18E − 05 | 92.7 | 1.2 |
| 1.01E − 04 | 98.9 | 8.2 |
| 3.18E − 04 | 98.6 | 15.6 |
| 1.00E − 03 | 78.8 | 9.5 |
| 3.17E − 03 | 81.3 | 4.7 |
| 1.00E − 02 | 66.4 | 8.4 |
| 3.17E − 02 | 56.3 | 4.2 |
| 1.00E − 01 | 51.6 | 3.3 |
| 3.16E − 01 | 47.3 | 6.9 |
| 1.00E + 00 | 49.8 | 0.7 |

U-87 MG
Human brain cancer (glioblastoma)

| | Relative cell count (%) | |
|---|---|---|
| Concentration (microM) | Mean | StdDev |
| 3.18E − 05 | 108.2 | 10.2 |
| 1.01E − 04 | 101.1 | 0.6 |
| 3.18E − 04 | 85.3 | 5.8 |
| 1.00E − 03 | 78.8 | 1.6 |
| 3.17E − 03 | 71.4 | 5.9 |
| 1.00E − 02 | 70.4 | 13.2 |
| 3.17E − 02 | 60.4 | 2.7 |
| 1.00E − 01 | 52.1 | 2.9 |
| 3.16E − 01 | 50.7 | 1.5 |
| 1.00E + 00 | 48.9 | 3.9 |

U-118 MG
Human glioblastoma

| | Relative cell count (%) | |
|---|---|---|
| Concentration (microM) | Mean | StdDev |
| 3.18E − 05 | 105.5 | 15.7 |
| 1.01E − 04 | 109.0 | 8.9 |
| 3.18E − 04 | 77.1 | 8.3 |
| 1.00E − 03 | 65.7 | 0.7 |
| 3.17E − 03 | 63.4 | 0.2 |
| 1.00E − 02 | 64.8 | 1.5 |
| 3.17E − 02 | 61.2 | 6.8 |
| 1.00E − 01 | 60.0 | 1.5 |
| 3.16E − 01 | 60.8 | 3.0 |
| 1.00E + 00 | 71.6 | 18.9 |

A summary of this data regarding cell lines for which xenograft models are available is provided in Table 5:

TABLE 5

| Cell Line | Tissue | Type | EC₅₀ | Comment |
|---|---|---|---|---|
| A431 | Skin | Head/Neck | 1.2 × 10⁻² | Negative |
| | | | | control |
| OVACAR3 | Female GU | Ovarian | 4.3 × 10⁻² | mTOR over- |
| | | | | expressing |
| NCl-H510A | Lung | Small cell lung | 7.2 × 10⁻⁵ | mTOR over- |
| | | cancer (SCLC) | | expressing |
| SK-MEL-82 | Skin | Melanoma | 4.8 × 10⁻³ | mTOR over- |
| | | | | expressing |
| U-118 MG | Central Nervous | Glioblastoma | 2.9 × 10⁻⁴ | mTOR |
| | System (CNS) | | | mutation/ |
| | | | | missense |
| AsPC-1 | Pancreas | Pancreatic | 8.6 × 10⁻³ | rictor over- |
| | | | | expressing |
| SCaBER | Bladder | Bladder | 2.5 × 10⁻⁴ | rictor over- |
| | | | | expressing |
| U-138 MG | CNS | Glioblastoma | 1.4 × 10⁻³ | raptor over- |
| | | | | expressing, |
| | | | | potentially |
| | | | | non- |
| | | | | tumorigenic |

### Example 3

This example demonstrates that the polyketide of the present invention has unexpectedly advantageous pharmacokinetics compared to the polyketide disclosed in U.S. Pat. No. 9,382,266. Table 6 provides the pharmacokinetic parameters of Example (I) in male Sprague Dawley rats after a single bolus intravenous dose of 2 mg/kg.

TABLE 6

| Parameter | Unit | Value |
|---|---|---|
| Lambda_z | 1/h | 0.270 |
| t_{1/2} | h | 2.57 |
| Tmax (time maximum blood conc.) | h | 0.0333 |
| Cmax (maximum blood | | |
| concentration) | ng/ml | 912 |
| C₀ | ng/ml | 1563 |
| Cₗₐₛₜ_observed/Cₘₐₓ | | 0.0223 |
| AUC 0-t | ng/ml*h | 694 |
| AUC 0-infinity_observed | ng/ml*h | 769 |
| AUC 0-t/0-infinity observed | | 0.902 |
| AUMC 0-inf_observed | ng/ml*h\hat{ }2 | 2358 |
| MRT 0-infinity_observed | h | 3.06 |
| Vz_obs | (mg/kg)/(ng/ml) | 0.00963 |
| Cl_obs | (mg/kg)/(ng/ml)/h | 0.00260 |
| Vss_obs | (mg/kg)/(ng/ml) | 0.00797 |

Table 7 provides the pharmacokinetic parameters of Example I in male Sprague Dawley rats after a single bolus oral dose of 10 mg/kg.

TABLE 7

| Parameter | Unit | Value |
|---|---|---|
| Lambda_z | 1/h | 0.127 |
| t_{1/2} | h | 5.46 |
| Tₘₐₓ | h | 2 |
| Cₘₐₓ | ng/ml | 206 |
| T_{lag} | h | 0 |
| Cₗₐₛₜ_observed/Cₘₐₓ | | 0.062 |
| AUC 0-t | ng/ml*h | 1719 |
| AUC 0-infinity_observed | ng/ml*h | 1820 |
| AUC 0-t/0-inf_obs | | 0.945 |
| AUMC 0-inf_obs | ng/ml*h\hat{ }2 | 13410 |
| MRT 0-inf_obs | h | 7.37 |
| Vz/F_obs | (mg/kg)/(ng/ml) | 0.0433 |
| Cl/F_obs | (mg/kg)/(ng/ml)/h | 0.0055 |
| % F | | 0.47 |

This example demonstrates that the polyketide of Formula II has, in addition to a more balanced TORC1/TORC2 selectivity, an unexpectedly desirable pharmacokinetics including but not limited to a high oral bioavailability.

### Example 4

This example illustrates one method for determining the pharmacokinetics and bioavailability of the compound of the invention.

A person of skill in the art will be able to determine the pharmacokinetics and bioavailability of the compound of the invention using in vivo and in vitro methods known to a person of skill in the art, including but not limited to those described below and in Gallant-Haidner et al, 2000 and Trepanier et al, 1998 and references therein. The bioavailability of a compound is determined by a number of factors, (e.g. water solubility, cell membrane permeability, the extent of protein binding and metabolism and stability) each of which may be determined by in vitro tests as described in the examples herein, it will be appreciated by a person of skill in the art that an improvement in one or more of these factors will lead to an improvement in the bioavailability of a compound. Alternatively, the bioavailability of the compound of the invention may be measured using in vivo methods as described in more detail below, or in the examples herein.

In order to measure bioavailability in vivo, a compound may be administered to a test animal (e.g. mouse or rat) both intraperitoneally (i.p.) or intravenously (i.v.) and orally (p.o.) and blood samples are taken at regular intervals to examine how the plasma concentration of the drug varies over time. The time course of plasma concentration over time can be used to calculate the absolute bioavailability of the compound as a percentage using standard models. An example of a typical protocol is described below.

For example, mice or rats are dosed with 1 or 3 mg/kg of the compound of the invention i.v. or 1, 5 or 10 mg/kg of the compound of the invention p.o. Blood samples are taken at 5 min, 15 min, 1 h, 4 h and 24 h intervals, and the concentration of the compound of the invention in the sample is determined via LCMS-MS. The time-course of plasma or whole blood concentrations can then be used to derive key parameters such as the area under the plasma or blood concentration-time curve (AUC—which is directly proportional to the total amount of unchanged drug that reaches the systemic circulation), the maximum (peak) plasma or blood drug concentration, the time at which maximum plasma or blood drug concentration occurs (peak time), additional factors which are used in the accurate determination of bioavailability include: the compound's terminal half-life, total body clearance, steady-state volume of distribution and F %. These parameters are then analyzed by non-compartmental or compartmental methods to give a calculated percentage bioavailability, for an example of this type of method see Gallant-Haidner et al, 2000 and Trepanier et al, 1998, and references therein.

Shown below are whole blood concentrations following administration of the compound of Formula II to mice. The compound of Formula II was administered to mice at 2 mg/kg or 10 mg/kg and the concentration of the compound in whole blood determined. These determinations were made using protein precipitation, liquid chromatography (LC), and mass spectrometry (MS/MS). Ten μl aliquots of whole blood and matrix calibration standards were distributed in a 96-well plate; 10 μl aliquots of blank matrix for matrix blanks and control blanks were included as controls. Ten μl of water was added to each sample followed by vortexing. One hundred sixty ml of internal standard was added to each sample except the matrix blanks; 160 μl 70:30 water:acetonitrile (ACN) was added to matrix blanks. This was followed by a five-minute vortex at >3500 rpm. One hundred fifty μl of the resultant supernatant was then transferred to a new 96-well plate and the samples blown to dryness at 35° C. The resultant product was then reconstituted with 90 μl ACN. LC was carried out using the equipment, conditions and calibration standards are shown in Tables 8 and 9.

TABLE 8

| | | Time | % | Flow |
|---|---|---|---|---|
| | | (sec) | MPB | (mL/min) |
| LC Conditions: | | | | |
| Column Id. & Dimensions: | Waters HSS T3, 30 × 2.1 mm | | | |
| Temperature (° C.) | Ambient | 15 | 65 | 0.800 |
| Mobile Phase A | 0.1% Formic Acid in Water | 75 | 85 | 0.800 |
| Mobile Phase B | 0.1% Formic Acid in Acetonitrile | 5 | 95 | 0.800 |
| Needle Rinse 1 | 25:25:25:25:0.1 MeOH:H2O:ACN:IPA:NH4OH | 25 | 95 | 0.800 |
| Needle Rinse 2 | 10:90:0.1 MeOH:H2O:FA | 30 | 65 | 0.800 |

| | |
|---|---|
| MS Conditions | |
| MS/MS: | API-5500 |
| Ionization Method: | Electrospray |
| Positive/Negative Ion: | Positive |
| Resolution: | Unit |
| Source Temperature | 550 |
| (° C.): | |
| Transitions (m/z): | Formula II: 918.5/409.3 Da // Int Std d4-AEA: 352.1/66.0 Da |

TABLE 9
Summary of Calibration Standard Back-Calculated
Concentrations of Formula II in Mouse Whole Blood

| | STD | | | | | | | | | | |
|---|---|---|---|---|---|---|---|---|---|---|---|
| | 1 | 2 | 3 | 4 | 5 | 6 | 7 | 8 | 9 | 10 | 11 |
| | Nominal Concentration (ng/mL) | | | | | | | | | | |
| | 1.00 | 2.00 | 5.00 | 10.0 | 25.0 | 50.0 | 100 | 200 | 500 | 800 | 1000 |
| | NR | NR | 4.78 | 8.03 | 23.3 | 52.3 | 102 | 216 | 402 | 773 | 946 |
| | NR | NR | 5.44 | 11.3 | 24.9 | 49.2 | 90.4 | 229 | 539 | 841 | 1060 |
| Mean | NA | NA | NA | 9.67 | 24.1 | 50.8 | 96.2 | 223 | 471 | 807 | 1003 |
| SD | NA | NA | NA | 2.31 | 1.13 | 2.19 | 8.20 | 9.19 | 96.9 | 48.1 | 80.6 |
| % Bias | NA | NA | NA | −3.4 | −3.6 | 1.5 | −3.8 | 11.3 | −5.9 | 0.9 | 0.3 |
| n | 0 | 0 | 2 | 2 | 2 | 2 | 2 | 2 | 2 | 2 | 2 |

N/A = Not Applicable // NR = Not Reported

The results of these analyses are summarized in Tables 10 and 11.

TABLE 10

| | Formula II Concentrations | | | | | |
|---|---|---|---|---|---|---|
| Group 3 (2 mg/kg) | (ng/mL) in Mouse Whole Blood | | | | | |
| IP (QD × 3 days)—Day 1 | Animal ID | | | | | |
| Time Points (hrs) | 7 | 8 | 9 | Mean | SD | % CV |
| 1.00 | 452 | 380 | 317 | 383 | 67.5 | 17.6% |
| 2.00 | 415 | 415 | 378 | 403 | 21.4 | 5.3% |
| 6.00 | 382 | 520 | 490 | 464 | 72.6 | 15.6% |
| 24.0 | 12.2 | 18.4 | 14.7 | 15.1 | 3.12 | 20.7% |
| Day 2 | | | | | | |
| 1.00 | 481 | 208 | 524 | 404 | 171 | 42.4% |
| 2.00 | 501 | 233 | 570 | 435 | 178 | 41.0% |
| 6.00 | 553 | 632 | 552 | 579 | 45.9 | 7.9% |
| 24.0 | 15.3 | 80.6 | 35.2 | 43.7 | 33.5 | 76.6% |
| Day 3 | | | | | | |
| 1.00 | 628 | 726 | 600 | 651 | 66.2 | 10.2% |
| 2.00 | 478 | 489 | 535 | 501 | 30.2 | 6.0% |
| 6.00 | 416 | 745 | 604 | 588 | 165 | 28.1% |
| 24.0 | 17.6 | 59.6 | 48.6 | 41.9 | 21.8 | 51.9% |

TABLE 11

| | Formula II Concentrations | | | | | |
|---|---|---|---|---|---|---|
| Group 4 (10 mg/kg) | (ng/mL) in Mouse Whole Blood | | | | | |
| IP (QD × 3 days)—Day 1 | Animal ID | | | | | |
| Time Points (hrs) | 10 | 11 | 12 | Mean | SD | % CV |
| 1.00 | 560 | 641 | 769 | 657 | 105 | 16.0% |
| 2.00 | 793 | 869 | 503 | 722 | 193.1 | 26.8% |
| 6.00 | 1000 | 1950 | 544 | 1165 | 717 | 61.6% |
| 24.0 | 222 | 181 | 210 | 204 | 21.1 | 10.3% |
| Day 2 | | | | | | |
| 1.00 | 3410 | 2970 | 546 | 2309 | 1542 | 66.8% |
| 2.00 | 3670 | 3930 | 2370 | 3323 | 836 | 25.1% |
| 6.00 | 2770 | 3390 | 609 | 2256 | 1460 | 64.7% |
| 24.0 | 123 | 336 | 78.0 | 179 | 138 | 77.0% |
| Day 3 | | | | | | |
| 1.00 | AQL | 3420 | 987 | 2204 | NA | NA |
| 2.00 | 5170 | 3650 | 777 | 3199 | 2231 | 69.7% |
| 6.00 | 3330 | 2890 | 730 | 2317 | 1392 | 60.1% |
| 24.0 | 223 | 323 | 124 | 223 | 99.5 | 44.6% |

AQL: Original value above quantiation limit (1000 ng/mL) Sample was diluted prior to re-analysis, but due to a sequence error the samples will be re-injected.

As shown above, Formula II exhibits sufficient concentrations in whole blood over time following administration at 2 mg/kg or 10 mg/kg via the intraperitoneal route (IP) once daily (QD) for three days.

### Example 5

### Xenograft Studies

A study was conducted to determine the anti-tumor efficacy of the compound of Formula II on U-118 MG (ATCC® HTB-15, human brain glioblastoma) solid tumors in female nude mice. In this study, advanced-stage subcutaneous xenografts were established to evaluate the antitumor activity of test agents so that clinically relevant parameters of activity could be determined. The end point used to assess drug efficacy was relative tumor growth (comparing tumors in treated versus control mice). In these models, tumor growth is monitored and test agent treatment is typically initiated once tumors reach a weight range of 100-300 mg. Tumor size and body weights were obtained two times per week for determination of toxicity and efficacy. The U-118 MG (ATCC® HTB-15) cell line used in this study was isolated from a malignant glioblastoma taken from a 50-year-old male Caucasian. Study endpoints were determined using the parameters: percent tumor growth inhibition (% TGI)=100 (Wc−Wt)/Wc=100(1−Wt/Wc), where We is the Median tumor weight of control group and Wt is the Median tumor weight of the treated group; tumor remission and regression (% REG=100(W0−Wi)W0, where WO is the Median tumor weight for treated group at the initiation of treatment and Wi is the Median tumor weight for that group at any given time; log₁₀ cell kill=[T−C value in days/(3.32) (Td)], where T−C is the tumor growth delay; the T=Median time (in days) required for the treatment group tumors to reach a predetermined size (i.e., 1,000 mg) and C is the Median time (in days) for the control group to reach the same size; and, tumor-free survivors are excluded from these calculations, where Td=the median tumor doubling time (in days) for the control group.

Female, athymic nude-Foxn1ₙᵤ mice (5-6 weeks old weighing approximately 19-23 grams (mean approx. 21 g) at study initiation (Day 1) (Envigo, Indianapolis, Ind.)) were identified by tail tattoo and housed separately (5 per cage) in Optimax polycarbonate cages with polycarbonate tops, irradiated corn cob bedding, and suspended food and water bottles. During the acclimation and study periods, animals were housed in a laboratory environment with temperatures ranging 67-76° F. and relative humidity of 30%-70%. Automatic timers provided 12 hours of light and 12 hours of dark. Animals were allowed access ad libitum to sterile Harlan Teklad Rodent Chow and sterile, pH 3.0 water. U-118 MG (ATCC® HTB-15) tumor cells were grown in tissue culture and expanded to implant 3×10⁶ cells subcutaneously (SC) in serum-free growth medium on the flank of the mice. Tumor growth was monitored daily. When calculated tumor volume reached approximately 100-300 mm³ (or 100-300 mg), tumor-bearing mice were weighed and randomized into treatment groups. Treatment was initiated after randomization (Study Day 1) and continued as indicated (10 mice per group, QD, 10 ml/kg, either Vehicle Control or Formula II at 10 mg/kg (as a suspension in 2% ethanol, 40% polyethylene glycol 400 (PEG 400), and 58% saline (prepared by dissolving the compound first in 2% ethanol, then adding PEG and saline); suspension prepared every two weeks and frozen at −20° C. between uses). Tumor growth and body weight was measured twice weekly, and animals were observed daily for signs of toxicity and tumor ulceration. Tumor measurements were taken along the length and width using vernier calipers, and tumor volumes were calculated using the following formula: (L×W²)/2. Tumor volume (absolute and percent of baseline) and body weight measurements were compared to vehicle controls using a one-way analysis of variance (ANOVA) with a Dunnett's multiple comparison post-hoc analysis. Significance was set at p≤0.05. Blood samples were collected on Days 16 and 30 from animals 1-5 in Groups 2-4. Whole blood (K₂EDTA, 50 μl/mouse) was collected pre-dose on Day 16 and 24 hours post-dose on Day 30 (trough levels) via retro-orbital blood draw and stored frozen at −80° C. Animals were kept alive after the scheduled study termination date (30 days post treatment initiation) to continue weighing and tumor measurements for evaluation of a possible vehicle effect on tumor growth.

Mice with subcutaneously (SC) implanted tumors were dosed daily (QD) on Days 1-29 by the intraperitoneal (IP) route with vehicle (2% ethanol (EtOH (Sigma))/40% PEG 400 (Sigma)/58% saline (VetPath)) or the compound of Formula II (10 mg/kg). Tumor growth and body weights were measured twice weekly as described above, and animals were observed daily for signs of toxicity and tumor ulceration. Efficacy evaluation was based on disease progression after treatment (durable cures), tumor volume, and body weight measurements.

As summarized in Table 12, there were no significant differences in body weight following administration of either vehicle control or a compound of Formula II after 30 days. One observed difference is that animals treated with the compound of Formula II gained a mean 3.13% body weight as compared to only 2.26% for the vehicle control.

TABLE 12

| Group 1 | Day 30 | | Change in |
|---|---|---|---|
| Vehicle | Body | Body Wt. % | Body |
| Control | Weight | Δ from | Weight |
| IP, UD | (g) | Baseline | Day 1-30 |
| 1 | 20.44 | 9.89% | 1.84 |
| 2 | 20.48 | 8.47% | 1.60 |
| 3 | 21.22 | 11.80% | 2.24 |
| 4 | 21.48 | 13.05% | 2.48 |
| 5 | 22.02 | 14.87% | 2.85 |
| 6 | 21.80 | 13.07% | 2.52 |
| 7 | 21.47 | 11.01% | 2.13 |
| 8 | 21.28 | 9.63% | 1.87 |
| 9 | 22.32 | 13.88% | 2.72 |
| 10 | 21.97 | 11.92% | 2.34 |
| Mean | 21.45 | 11.76% | 2.26 |
| Median | 21.48 | 11.86% | 2.29 |
| SE | 0.20 | 0.64% | 0.13 |
| Percent Survival | 100% | 100% | |
| | Day 30 | | Change in |
| Group 2 | Body | Body Wt. % | Body |
| Formula I | Weight | Δ from | Weight |
| IP, UD | (g) | Baseline | Day 1-30 |
| 1 | 22.55 | 7.95% | 1.66 |
| 2 | 24.34 | 16.07% | 3.37 |
| 3 | 24.25 | 15.37% | 3.23 |
| 4 | 23.98 | 13.92% | 2.93 |
| 5 | 23.95 | 13.61% | 2.87 |
| 6 | 25.49 | 20.24% | 4.29 |
| 7 | 25.97 | 22.44% | 4.76 |
| 8 | 23.14 | 8.64% | 1.84 |
| 9 | 23.24 | 8.80% | 1.88 |
| 10 | 26.83 | 19.72% | 4.42 |
| Mean | 24.37 | 14.68% | 3.13 |
| Median | 24.12 | 14.64% | 3.08 |
| SE | 0.43 | 1.62% | 0.35 |
| ANOVA | | 0.337 | 0.127 |
| (Dunnett's) vs. | | | |
| Group 1 | | | |
| Percent Survival | 100% | 100% | |

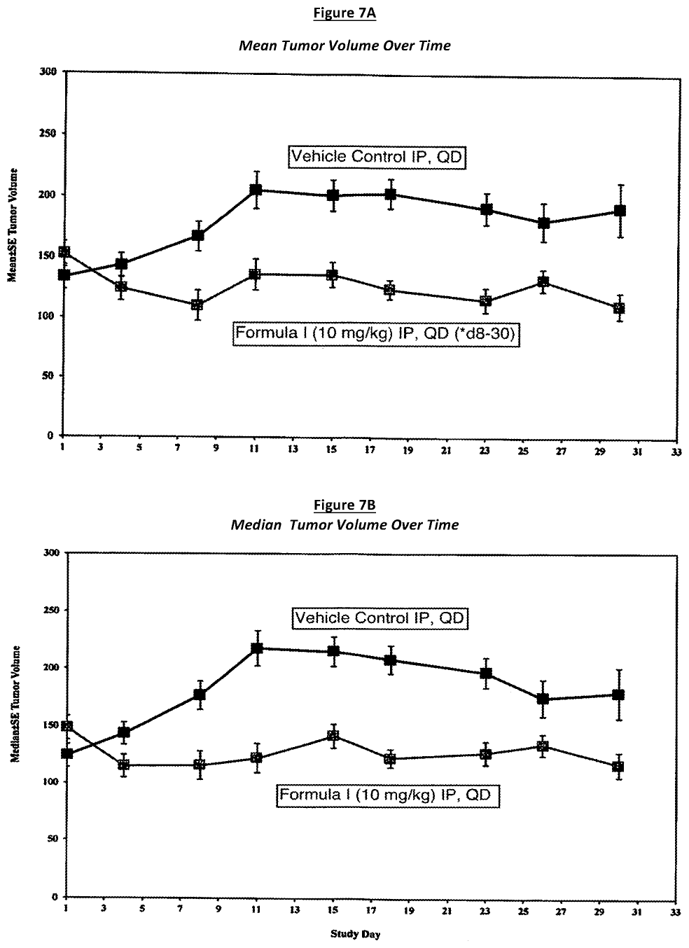

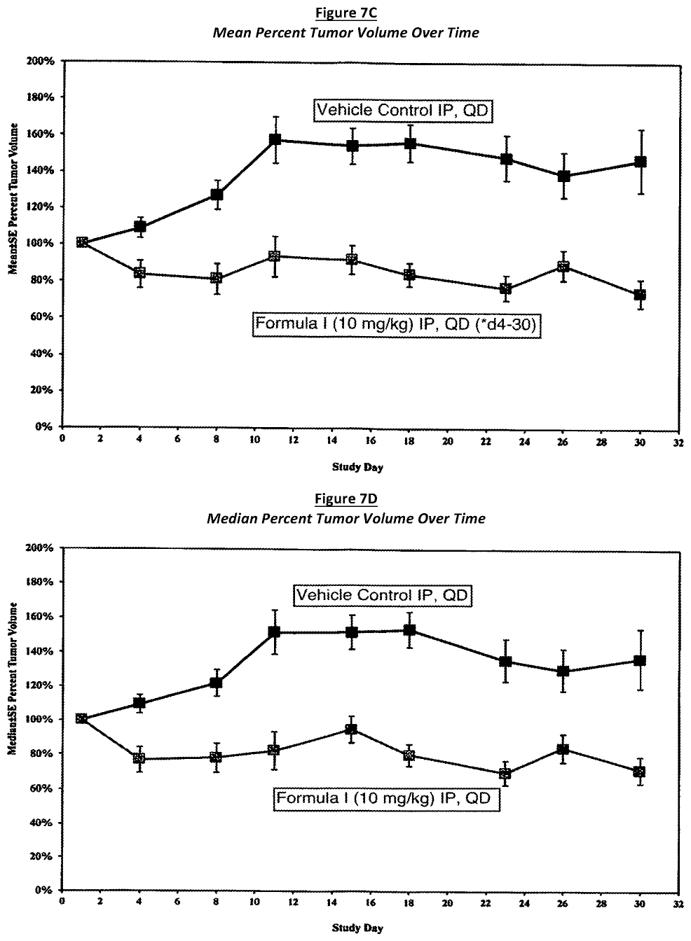

The data presented in Tables 13-19 and FIGS. 7A-D shows that administration of the compound of Formula II was effective in reducing U118 tumor growth, with the effects on tumor volume expressed as a percent of baseline being observed as early as day four of treatment and the effect on mean tumor volume being observed as early as day 8. The data is presented in the Tables 13-19 and FIGS. 7A-D.

TABLE 13

| Vehicle | Day 1 | | | Day 4 | | | |
|---|---|---|---|---|---|---|---|
| Control | Length | Width | Tumor | Length | Width | Tumor | % Tumor |
| IP, QD | (mm) | (mm) | Vol. (mm²) | (mm) | (mm) | Vol. (mm³) | Volume |
| 1 | 9.70 | 4.67 | 105.77 | 9.41 | 4.44 | 92.75 | 88% |
| 2 | 9.68 | 6.57 | 208.92 | 10.30 | 6.41 | 211.60 | 101% |
| 3 | 11.81 | 4.47 | 117.99 | 11.56 | 4.98 | 143.35 | 121% |
| 4 | 9.54 | 5.09 | 123.58 | 10.47 | 4.88 | 124.67 | 101% |
| 5 | 7.76 | 5.81 | 130.97 | 9.71 | 5.76 | 161.08 | 123% |
| 6 | 8.35 | 4.95 | 102.30 | 8.96 | 5.31 | 126.32 | 123% |
| 7 | 7.36 | 6.22 | 142.37 | 7.46 | 5.89 | 129.40 | 91% |
| 8 | 7.54 | 5.48 | 113.21 | 7.98 | 6.18 | 152.39 | 135% |
| 9 | 8.31 | 6.29 | 164.39 | 7.95 | 6.01 | 143.58 | 87% |
| 10 | 7.76 | 5.67 | 124.74 | 7.22 | 6.37 | 146.48 | 117% |
| Mean | | | 133.42 | | | 143.16 | 109% |
| Median | | | 124.16 | | | 143.46 | 109% |
| SE | | | 10.16 | | | 9.69 | 5% |
| | Day 1 | | | Day 4 | | | |
| Formula II | Length | Width | Tumor | Length | Width | Tumor | % Tumor |
| IP, QD | (mm) | (mm) | Vol. (mm²) | (mm) | (mm) | Vol. (mm³) | Volume |
| 1 | 6.80 | 6.23 | 131.96 | 8.23 | 6.47 | 172.26 | 131% |
| 2 | 8.50 | 6.51 | 180.12 | 8.61 | 5.06 | 110.22 | 61% |
| 3 | 9.46 | 5.18 | 126.92 | 8.91 | 5.36 | 127.99 | 101% |
| 4 | 9.48 | 6.51 | 200.88 | 9.80 | 5.66 | 156.97 | 78% |
| 5 | 8.35 | 6.08 | 154.33 | 8.74 | 4.78 | 99.85 | 65% |
| 6 | 9.16 | 6.57 | 197.70 | 6.86 | 5.90 | 119.40 | 60% |
| 7 | 8.16 | 6.17 | 155.32 | 9.22 | 6.06 | 169.30 | 109% |
| 8 | 8.45 | 5.81 | 142.62 | 8.29 | 4.81 | 95.90 | 67% |
| 9 | 7.83 | 5.89 | 135.82 | 6.46 | 5.63 | 102.38 | 75% |
| 10 | 7.65 | 5.04 | 97.16 | 7.31 | 4.82 | 84.91 | 87% |
| Mean | | | 152.28 | | | 123.92 | 83% |
| Median | | | 148.48 | | | 114.81 | 77% |
| SE | | | 10.37 | | | 10.03 | 7% |
| ANOVA | | | 0.377 | | | 0.327 | 0.012 |
| (Dunnett's) vs. | | | | | | | |
| Group 1 | | | | | | | |

TABLE 14

| Group 1 | | | | | | | | |
|---|---|---|---|---|---|---|---|---|
| Vehicle | Day 8 | | | | Day 11 | | | |
| Control | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 9.37 | 5.02 | 118.06 | 112% | 11.20 | 4.45 | 110.89 | 105% |
| 2 | 9.51 | 7.08 | 238.35 | 114% | 10.42 | 6.89 | 247.33 | 118% |
| 3 | 11.71 | 5.62 | 184.93 | 157% | 12.40 | 6.31 | 246.86 | 209% |
| 4 | 9.84 | 4.60 | 104.11 | 84% | 9.79 | 5.36 | 140.63 | 114% |
| 5 | 10.13 | 6.06 | 186.01 | 142% | 9.90 | 7.17 | 254.47 | 194% |
| 6 | 10.04 | 5.90 | 174.75 | 171% | 9.56 | 6.84 | 223.64 | 219% |
| 7 | 9.04 | 6.28 | 178.26 | 125% | 9.06 | 6.82 | 210.70 | 148% |
| 8 | 8.92 | 5.49 | 134.42 | 119% | 8.46 | 6.45 | 175.98 | 155% |
| 9 | 7.69 | 6.87 | 181.47 | 110% | 9.05 | 7.16 | 231.98 | 141% |
| 10 | 7.51 | 6.73 | 170.07 | 136% | 8.28 | 7.15 | 211.65 | 170% |
| Mean | | | 167.04 | 127% | | | 205.41 | 157% |
| Median | | | 176.50 | 122% | | | 217.64 | 152% |
| SE | | | 12.29 | 8% | | | 15.27 | 13% |
| Group 2 | Day 8 | | | | Day 11 | | | |
| Formula II | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 7.64 | 5.31 | 107.71 | 82% | 8.11 | 6.55 | 173.97 | 132% |
| 2 | 6.88 | 6.24 | 133.95 | 74% | 8.36 | 6.34 | 168.02 | 93% |
| 3 | 10.26 | 5.28 | 143.02 | 113% | 10.85 | 5.55 | 167.10 | 132% |
| 4 | 8.22 | 5.10 | 106.90 | 53% | 8.26 | 5.34 | 117.77 | 59% |
| 5 | 8.23 | 5.77 | 137.00 | 89% | 7.85 | 4.90 | 94.24 | 61% |
| 6 | 7.40 | 5.78 | 123.61 | 63% | 6.79 | 5.68 | 109.53 | 55% |
| 7 | 9.00 | 6.83 | 209.92 | 135% | 8.65 | 6.97 | 210.11 | 135% |
| 8 | 8.27 | 4.84 | 96.86 | 68% | 8.44 | 4.73 | 94.41 | 66% |
| 9 | 6.06 | 4.74 | 68.08 | 50% | 6.22 | 5.58 | 96.83 | 71% |
| 10 | 6.38 | 5.06 | 81.68 | 84% | 8.27 | 5.53 | 126.45 | 130% |
| | | | 0.00 | | | | | |
| Mean | | | 109.88 | 81% | | | 135.84 | 93% |
| Median | | | 115.66 | 78% | | | 122.11 | 82% |
| SE | | | 12.53 | 8% | | | 12.93 | 11% |
| ANOVA | | | 0.014 | 0.000 | | | 0.004 | 0.003 |
| (Dunnett's) | | | | | | | | |
| vs. Group 1 | | | | | | | | |

TABLE 15

| Group 1 | | | | | | | | |
|---|---|---|---|---|---|---|---|---|
| Vehicle | Day 15 | | | | Day 18 | | | |
| Control | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 10.01 | 5.09 | 129.67 | 123% | 12.20 | 4.99 | 151.89 | 144% |
| 2 | 10.95 | 6.55 | 234.89 | 112% | 10.08 | 7.01 | 247.67 | 119% |
| 3 | 12.39 | 6.25 | 241.99 | 205% | 10.65 | 6.29 | 210.68 | 179% |
| 4 | 10.00 | 5.58 | 155.68 | 126% | 10.49 | 5.40 | 152.94 | 124% |
| 5 | 11.55 | 6.34 | 232.13 | 177% | 12.11 | 6.46 | 252.68 | 193% |
| 6 | 9.60 | 5.81 | 162.03 | 158% | 9.62 | 5.90 | 167.44 | 164% |
| 7 | 9.67 | 6.26 | 189.47 | 133% | 9.35 | 6.17 | 177.97 | 125% |
| 8 | 8.81 | 6.83 | 205.49 | 182% | 9.38 | 7.19 | 242.45 | 214% |
| 9 | 9.86 | 6.96 | 238.82 | 145% | 9.46 | 6.88 | 223.89 | 136% |
| 10 | 8.56 | 7.24 | 224.35 | 180% | 8.54 | 6.94 | 205.66 | 165% |
| Mean | | | 201.45 | 154% | | | 203.33 | 156% |
| Median | | | 214.92 | 152% | | | 208.17 | 154% |
| SE | | | 12.73 | 10% | | | 12.26 | 10% |
| Group 2 | Day 15 | | | | Day 18 | | | |
| Formula II | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 8.36 | 6.38 | 170.14 | 129% | 8.04 | 5.69 | 130.15 | 99% |
| 2 | 8.21 | 5.12 | 107.61 | 60% | 8.23 | 5.35 | 117.78 | 65% |
| 3 | 11.45 | 4.70 | 126.47 | 100% | 10.05 | 5.06 | 128.66 | 101% |
| 4 | 8.75 | 4.87 | 103.76 | 52% | 9.12 | 5.21 | 123.78 | 62% |
| 5 | 8.42 | 5.74 | 138.71 | 90% | 7.79 | 5.63 | 123.46 | 80% |
| 6 | 8.05 | 6.37 | 163.32 | 83% | 7.36 | 5.74 | 121.25 | 61% |
| 7 | 8.99 | 6.24 | 175.02 | 113% | 9.03 | 6.47 | 189.00 | 122% |
| 8 | 9.68 | 5.72 | 158.36 | 111% | 8.79 | 4.87 | 104.24 | 73% |
| 9 | 7.00 | 6.43 | 144.71 | 107% | 6.52 | 5.77 | 108.53 | 80% |
| 10 | 6.08 | 4.91 | 73.29 | 75% | 6.42 | 5.42 | 94.30 | 97% |
| Mean | | | 136.14 | 92% | | | 124.11 | 84% |
| Median | | | 141.71 | 95% | | | 122.35 | 80% |
| SE | | | 10.48 | 8% | | | 8.06 | 6% |
| ANOVA | | | 0.001 | 0.000 | | | 0.000 | 0.000 |
| (Dunnett's) | | | | | | | | |
| vs. Group 1 | | | | | | | | |

TABLE 16

| Group 1 | | | | | | | | |
|---|---|---|---|---|---|---|---|---|
| Vehicle | Day 23 | | | | Day 26 | | | |
| Control | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 12.08 | 4.86 | 142.66 | 135% | 11.80 | 5.68 | 190.35 | 180% |
| 2 | 11.02 | 6.65 | 243.67 | 117% | 12.12 | 6.75 | 276.11 | 132% |
| 3 | 10.06 | 6.57 | 217.12 | 184% | 11.34 | 5.16 | 150.97 | 128% |
| 4 | 9.91 | 5.09 | 128.37 | 104% | 8.40 | 4.98 | 104.16 | 84% |
| 5 | 12.54 | 6.04 | 228.74 | 175% | 11.69 | 6.36 | 236.43 | 181% |
| 6 | 9.68 | 6.41 | 198.87 | 194% | 10.21 | 6.33 | 204.55 | 200% |
| 7 | 9.80 | 6.30 | 194.48 | 137% | 9.97 | 5.84 | 170.02 | 119% |
| 8 | 9.71 | 7.00 | 237.90 | 210% | 9.94 | 6.01 | 179.52 | 159% |
| 9 | 7.74 | 6.76 | 176.85 | 108% | 8.20 | 6.36 | 165.84 | 101% |
| 10 | 7.51 | 6.24 | 146.21 | 117% | 6.68 | 6.24 | 130.05 | 104% |
| Mean | | | 191.49 | 148% | | | 180.80 | 139% |
| Median | | | 196.67 | 136% | | | 174.77 | 130% |
| SE | | | 13.17 | 12% | | | 15.82 | 12% |
| Group 2 | Day 23 | | | | Day 26 | | | |
| Formula II | Length | Width | Tumor | % Tumor | Length | Width | Tumor | % Tumor |
| IP, QD | (mm) | (mm) | Vol. (mm³) | Volume | (mm) | (mm) | Vol. (mm³) | Volume |
| 1 | 7.80 | 6.26 | 152.83 | 116% | 8.84 | 5.56 | 136.64 | 104% |
| 2 | 7.11 | 5.20 | 96.13 | 53% | 7.73 | 5.69 | 125.13 | 69% |
| 3 | 11.12 | 4.78 | 127.04 | 100% | 11.90 | 5.75 | 196.72 | 155% |
| 4 | 9.31 | 5.28 | 129.77 | 65% | 10.08 | 5.27 | 139.98 | 70% |
| 5 | 8.13 | 4.72 | 90.56 | 59% | 8.48 | 5.68 | 136.79 | 89% |
| 6 | 7.72 | 6.19 | 147.90 | 75% | 8.27 | 5.64 | 131.53 | 67% |
| 7 | 7.10 | 5.97 | 126.53 | 81% | 8.24 | 5.84 | 140.52 | 90% |
| 8 | 9.52 | 5.50 | 143.99 | 101% | 8.04 | 5.18 | 107.87 | 76% |
| 9 | 6.58 | 4.99 | 81.92 | 60% | 8.08 | 5.62 | 127.60 | 94% |
| 10 | 5.11 | 4.72 | 56.92 | 59% | 6.61 | 4.83 | 77.10 | 79% |
| Mean | | | 115.36 | 77% | | | 131.99 | 89% |
| Median | | | 126.78 | 70% | | | 134.09 | 84% |
| SE | | | 10.15 | 7% | | | 9.45 | 8% |
| ANOVA | | | 0.000 | 0.000 | | | 0.027 | 0.003 |
| (Dunnett's) | | | | | | | | |
| vs. Group 1 | | | | | | | | |

TABLE 17

| Group 1 | | | | |
|---|---|---|---|---|
| Vehicle | Day 30 | | | |
| Control | Length | Width | Tumor | %Tumor |
| IP, UD | (mm) | (mm) | Vol. (mm³) | Volume |
| 1 | 11.06 | 5.19 | 148.96 | 141% |
| 2 | 10.56 | 6.43 | 218.30 | 104% |
| 3 | 9.37 | 5.66 | 150.09 | 127% |
| 4 | 6.89 | 4.87 | 81.70 | 66% |
| 5 | 11.14 | 7.53 | 315.82 | 241% |
| 6 | 10.16 | 5.77 | 169.13 | 165% |
| 7 | 9.78 | 6.21 | 188.58 | 132% |
| 8 | 9.75 | 7.42 | 268.40 | 237% |
| 9 | 9.48 | 7.01 | 232.92 | 142% |
| 10 | 8.27 | 5.80 | 139.10 | 112% |
| Mean | | | 191.30 | 147% |
| Median | | | 178.85 | 137% |
| SE | | | 21.77 | 18% |
| Group 2 | Day 30 | | | |
| Formula II | Length | Width | Tumor | % Tumor |
| IP, QD | (mm) | (mm) | Vol. (mm³) | Volume |
| 1 | 8.53 | 5.81 | 143.97 | 109% |
| 2 | 7.94 | 5.00 | 99.25 | 55% |
| 3 | 8.12 | 5.53 | 124.16 | 98% |
| 4 | 9.26 | 5.36 | 133.02 | 66% |
| 5 | 8.54 | 5.75 | 141.18 | 91% |
| 6 | 6.93 | 4.83 | 80.83 | 41% |
| 7 | 7.74 | 6.23 | 150.21 | 97% |
| 8 | 9.44 | 4.80 | 108.75 | 76% |
| 9 | 6.48 | 5.11 | 84.60 | 62% |
| 10 | 5.65 | 3.84 | 41.66 | 43% |
| Mean | | | 110.76 | 74% |
| Median | | | 116.45 | 71% |
| SE | | | 10.93 | 8% |
| ANOVA | | | 0.001 | 0.000 |
| (Dun netts) | | | | |
| vs. Group 1 | | | | |

TABLE 18
Summary of Statistical Analysis (Tumor Volume)
Tumor Volume (Day 1)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | −18.86 | −52.03 to | No | ns | 0.3765 | | | |
| | | 14.31 | | | | | | |
| Test details | Mean 1 | Mean 2 | Mean Diff. | SE of diff. | n1 | n2 | q | DF |
| Vehicle Control vs. Formula II | 133.4 | 152.3 | −18.86 | 13.53 | 10 | 10 | 1.394 | 36 |

Tumor Volume (Day 4)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 19.24 | −12.53 to | No | ns | 0.3273 | | | |
| | | 51.01 | | | | | | |
| Test details | Mean 1 | Mean 2 | Mean Diff. | SE of diff. | n1 | n2 | q | DF |
| Vehicle Control vs. Formula II | 143.2 | 123.9 | 19.24 | 12.96 | 10 | 10 | 1.485 | 36 |

Tumor Volume (Day 8)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 46.17 | 8.186 to | Yes | * | 0.014 | | | |
| | | 84.16 | | | | | | |
| Test details | Mean 1 | Mean 2 | Mean Diff. | SE of diff. | n1 | n2 | q | DF |
| Vehicle Control vs. Formula II | 167 | 120.9 | 46.17 | 15.49 | 10 | 10 | 2.981 | 36 |

Tumor Volume (Day 11)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 69.57 | 20.84 to | Yes | ** | 0.0035 | | | |
| | | 118.3 | | | | | | |
| Test details | Mean 1 | Mean 2 | Mean Diff. | SE of diff. | n1 | n2 | q | DF |
| Vehicle Control vs. Formula II | 205.4 | 135.8 | 69.57 | 19.87 | 10 | 10 | 3.501 | 36 |

Tumor Volume (Day 15)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 65.31 | 24.35 to | Yes | ** | 0.0011 | | | |
| | | 106.3 | | | | | | |
| Test details | Mean 1 | Mean 2 | Mean Diff. | SE of diff. | n1 | n2 | q | DF |
| Vehicle Control vs. Formula II | 201.5 | 136.1 | 65.31 | 16.7 | 10 | 10 | 3.91 | 36 |

Tumor Volume (Day 18)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 79.21 | 42.49 to | Yes | **** | 0.0001 | | | |
| | | 115.9 | | | | | | |
| Test details | Mean 1 | Mean 2 | Mean Diff. | SE of diff. | n1 | n2 | q | DF |
| Vehicle Control vs. Formula II | 203.3 | 124.1 | 79.21 | 14.97 | 10 | 10 | 5.29 | 36 |

Tumor Volume (Day 23)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 76.13 | 40.74 to | Yes | **** | 0.0001 | | | |
| | | 111.5 | | | | | | |
| Test details | Mean 1 | Mean 2 | Mean Diff. | SE of diff. | n1 | n2 | q | DF |
| Vehicle Control vs. Formula II | 191.5 | 115.4 | 76.13 | 14.43 | 10 | 10 | 5.276 | 36 |

Tumor Volume (Day 26)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 48.81 | 4.647 to | Yes | * | 0.0273 | | | |
| | | 92.98 | | | | | | |
| Test details | Mean 1 | Mean 2 | Mean Diff. | SE of diff. | n1 | n2 | q | DF |
| Vehicle Control vs. Formula II | 180.8 | 132 | 48.81 | 18.01 | 10 | 10 | 2.71 | 36 |

Tumor Volume (Day 30)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 80.54 | 32.34 to | Yes | *** | 0.0007 | | | |
| | | 128.7 | | | | | | |
| Test details | Mean 1 | Mean 2 | Mean Diff. | SE of diff. | n1 | n2 | q | DF |
| Vehicle Control vs. Formula II | 191.3 | 110.8 | 80.54 | 19.65 | 10 | 10 | 4.098 | 36 |

TABLE 19
Summary of Statistical Analysis (Tumor Percent)
Tumor Percent (Day 4)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.2533 | 0.04981 to | Yes | * | 0.0116 | | | |
| | | 0.4567 | | | | | | |
| Test details | Mean 1 | Mean 2 | Mean Diff. | SE of diff. | n1 | n2 | q | DF |
| Vehicle Control vs. Formula II | 1.088 | 0.8348 | 0.2533 | 0.08296 | 10 | 10 | 3.053 | 36 |

Tumor Percent (Day 8)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.4598 | 0.2085 to | Yes | *** | 0.0002 | | | |
| | | 0.7111 | | | | | | |
| Test details | Mean 1 | Mean 2 | Mean Diff. | SE of diff. | n1 | n2 | q | DF |
| Vehicle Control vs. Formula II | 1.27 | 0.8104 | 0.4598 | 0.1025 | 10 | 10 | 4.487 | 36 |

Tumor Percent (Day 11)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.6386 | 0.2046 to | Yes | ** | 0.0026 | | | |
| | | 1.073 | | | | | | |
| Test details | Mean 1 | Mean 2 | Mean Diff. | SE of diff. | n1 | n2 | q | DF |
| Vehicle Control vs. Formula II | 1.573 | 0.9348 | 0.6386 | 0.177 | 10 | 10 | 3.608 | 36 |

Tumor Percent (Day 15)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.6233 | 0.328 to | Yes | **** | 0.0001 | | | |
| | | 0.9186 | | | | | | |
| Test details | Mean 1 | Mean 2 | Mean Diff. | SE of diff. | n1 | n2 | q | DF |
| Vehicle Control vs. Formula II | 1.541 | 0.9182 | 0.6233 | 0.1204 | 10 | 10 | 5.176 | 36 |

Tumor Percent (Day 18)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.7212 | 0.4002 to | Yes | **** | 0.0001 | | | |
| | | 1.042 | | | | | | |
| Test details | Mean 1 | Mean 2 | Mean Diff. | SE of diff. | n1 | n2 | q | DF |
| Vehicle Control vs. Formula II | 1.561 | 0.8401 | 0.7212 | 0.1309 | 10 | 10 | 5.51 | 36 |

Tumor Percent (Day 23)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.7113 | 0.39 to | Yes | **** | 0.0001 | | | |
| | | 1.033 | | | | | | |
| Test details | Mean 1 | Mean 2 | Mean Diff. | SE of diff. | n1 | n2 | q | DF |
| Vehicle Control vs. Formula II | 1.48 | 0.7687 | 0.7113 | 0.131 | 10 | 10 | 5.429 | 36 |

Tumor Percent (Day 26)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.4957 | 0.1528 to | Yes | ** | 0.0031 | | | |
| | | 0.8385 | | | | | | |
| Test details | Mean 1 | Mean 2 | Mean Diff. | SE of diff. | n1 | n2 | q | DF |
| Vehicle Control vs. Formula II | 1.388 | 0.8923 | 0.4957 | 0.1398 | 10 | 10 | 3.545 | 36 |

Tumor Percent (Day 30)

| Dunnett's multiple comparisons | Mean | 95.00% Cl | | | | | | |
|---|---|---|---|---|---|---|---|---|
| test | Diff. | of diff. | Significant? | Summary | Adjusted P Value | | | |
| Vehicle Control vs. Formula II | 0.7291 | 0.3481 to | Yes | *** | 0.0001 | | | |
| | | 1.11 | | | | | | |
| Test details | Mean 1 | Mean 2 | Mean Diff. | SE of diff. | n1 | n2 | q | DF |
| Vehicle Control vs. Formula II | 1.468 | 0.7387 | 0.7291 | 0.1554 | 10 | 10 | 4.693 | 36 |

As shown in Tables 13-19 and FIGS. 7A-7D, the compound of Formula II significantly reduced tumor growth in the U188 xenograft model over the 30-day test period and showed effects as early as four days following administration of Formula II (intraperitoneal (IP) administration once per day (QD)) as compared to administration of the Vehicle Control. Tumor size increased in vehicle control mice through Day 11 and then generally maintained a mean range of 181-205 mm³ (139-157% increase from baseline) through Day 30. Tumor volume as percent of baseline (Day 1) was significantly reduced on Days 4-30 in mice treated with the compound of Formula II as compared to vehicle controls. Thus, treatment with the compound of Formula II showed significant inhibitory effect on U-118 MG solid tumor growth in female nude mice as determined by evaluation of tumor size over time. Survival for animals treated with Vehicle Control or Formula II was 100% at 30 days.

### Example 6

This example illustrates one method of making the polyketide of Formula II.

Feed starter exo-(1R,2S,4R,5S)-5-hydroxybicyclo[2.2.1]heptane-2-carboxylate was

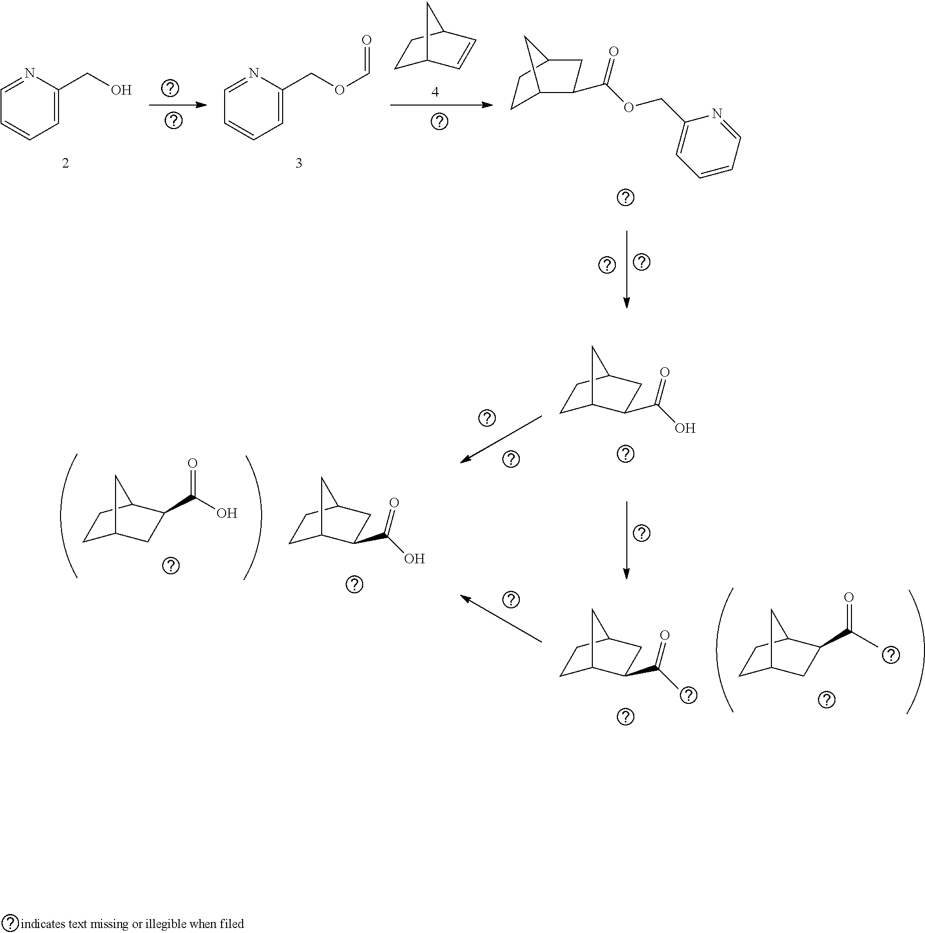

S. hygroscopicus BIOT-4010 or MG2-10 was cultured on medium 1 agar plates (see below) at 28° C. Spore stocks were prepared after growth on medium 1, preserved in 20% w/v glycerol:10% w/v lactose in distilled water and stored at −80° C. Vegetative cultures were prepared by inoculating 0.1 mL of frozen stock into 50 mL medium 2 (see below) in 250 mL flask. The culture was incubated for 36 to 48 hours at 28° C., 300 rpm.

A. Production Method

Vegetative cultures were inoculated at 2.5-5% v/v into medium 3. Cultivation was carried out for 6-7 days, 26° C., 300 rpm.

B. Feeding Procedure

The feeding/addition of Formula III was carried out 24-48 hours after inoculation and was fed at 1-2 mM final concentration unless stated otherwise.

Medium 1

| component | Source | Catalogue # | Per L |
|---|---|---|---|
| Corn steep powder | Sigma | C-8160 | 2.5 g |
| Yeast extract | Difco | 0127-17 | 3 g |
| Calcium carbonate | Sigma | C5929 | 3 g |
| Iron sulfate | Sigma | F8633 | 0.3 g |
| BACTO agar | | | 20 g |
| Wheat starch | Sigma | S2760 | 10 g |
| Water to | | | 1 L |

The media was then sterilized by autoclaving 121° C., 20 min.

MD6 Medium (Small Scale Fermentation Medium)

| Component | Per L |
|---|---|
| Toasted Nutrisoy (ADM Ingredients Ltd) | 30 g |
| Corn starch (Sigma) | 30 g |
| Avedex W80 dextrin (Deymer Ingredients Ltd) | 19 g |
| Yeast (Allinson) | 3 g |
| Corn Steep Solids (Sigma) | 1 g |
| KH2PO4 | 2.5 g |
| (NH4)2SO4 | 10 g |
| NaCl | 5 g |
| CaCO3 (Caltec) | 10 g |
| MnCl2•4H2O | 10 mg |
| MgSO4•7H2O | 2.5 mg |
| FeSO4•7H2O | 120 mg |
| ZnSO4•7H2O | 50 mg |
| MES (2-morpholinoethane sulfuric acid monohydrate) | 21.2 g |

pH is corrected to 6.0 with 1M NaOH

Before sterilization 0.4 mL of Sigma α-amylase (BAN 250) was added to 1 L of medium. Medium was sterilized for 20 min at 121° C. After sterilization 0.35 mL of sterile 40% fructose and 0.10 mL of L-lysine (140 mg/mL in water, filter-sterilized) was added to each 7 mL.

RapV7 Seed Medium

| Component | Per L |
|---|---|
| Toasted Nutrisoy (ADM Ingredients Ltd) | 5 g |
| Avedex W80 dextrin (Deymer Ingredients Ltd) | 35 g |
| Corn Steep Solids (Sigma) | 4 g |
| (NH4)2SO4 | 2 g |
| Lactic acid (80%) | 1.6 mL |
| CaCO3 (Caltec) | 7 g |

Adjust pH to 7.5 with 1M NaOH.

The media was then sterilized by autoclaving 121° C., 20 min. d-Glucose (to 10 g/L) was added after autoclaving.

MD6 Medium (Small Scale Fermentation Medium)

| Component | Per L |
|---|---|
| Toasted Nutrisoy (ADM Ingredients Ltd) | 30 g |
| Avedex W80 dextrin (Deymer Ingredients Ltd) | 19 g |
| Corn starch | 30 g |
| Yeast (Allinson) | 3 g |
| Corn Steep Solids (Sigma) | 1 g |
| KH2PO4 | 2.5 g |
| K2HPO4 | 2.5 g |
| (NH4)2SO4 | 10 g |
| NaCl | 5 g |
| CaCO3 (Caltec) | 10 g |
| MnC12•4H20 | 10 mg |
| MgSO4•7H20 | 2.5 mg |
| FeSO4•7H20 | 120 mg |
| ZnSO4•7H20 | 50 mg |
| MES | 21.2 g |

Medium was adjusted to pH6.0, 0.4 mL/L alpha-amylase (Sigma A7595-liquid, >250 units/g) added and the media sterilized for 30 min at 121° C. d-Fructose (to 20 g/L) and 1-lysine (monohydrochloride) (to 2 g/L) were added after autoclaving.

MD6/5-1 Medium (Medium Scale Fermentation Medium)

| Component | Per L |
|---|---|
| Toasted Nutrisoy (ADM Ingredients Ltd) | 15 g |
| Avedex W80 dextrin (Deymer Ingredients Ltd) | 50 g |
| Yeast (Allinson) | 3 g |
| Corn Steep Solids (Sigma) | 1 g |
| KH2PO4 | 2.5 g |
| K2HPO4 | 2.5 g |
| (NH4)2504 | 10 g |
| NaCI | 13 g |
| CaCO3 (Caltec) | 10 g |
| MnC12•4H2O | 3.5 mg |
| MgSO4•7H2O | 15 mg |
| FeSO4•7H2O | 150 mg |
| ZnSO4•7H2O | 60 mg |
| SAG 471 | 0.5 ml |

Medium was sterilized for 30 min at 121° C. After sterilization, 15 g of Fructose per L was added. After 48 hours, 0.5 g/L of L-lysine was added.

### Example 7

### Analytical Methods

Analytical Method A

Injection volume: 0.005-0.1 mL (as required depending on sensitivity). HPLC was performed on Agilent “Spherisorb” “Rapid Resolution” cartridges SB C8, 3 micron, 30 mm×2.1 mm, running a mobile phase of:
Mobile phase A: 0.01% Formic acid in pure water
Mobile phase B: 0.01% Formic acid in Acetonitrile
Flow rate: 1 mL/minute.
Linear gradient was used, from 5% B at 0 min to 95% B at 2.5 min holding at 95% B until 4 min returning to 5% B until next cycle. Detection was by UV absorbance at 254 nm and/or by mass spectrometry electrospray ionization (positive or negative) using a Micromass Quattro-Micro instrument.

Analytical Method B

Injection volume: 0.02 mL. HPLC was performed on 3 micron BDS C18 Hypersil (ThermoHypersil-Keystone Ltd) column, 150×4.6 mm, maintained at 50° C., running a mobile phase of:
Mobile phase A: Acetonitrile (100 mL), trifluoracetic acid (1 mL), 1 M ammonium acetate (10 mL) made up to 1 L with deionized water.
Mobile phase B: Deionized water (100 mL), trifluoracetic acid (1 mL), 1 M ammonium acetate (10 mL) made up to 1 L with acetonitrile.
Flow rate: 1 mL/minute.
A linear gradient from 55% B-95% B was used over 10 minutes, followed by 2 minutes at 95% B, 0.5 minutes to 55% B and a further 2.5 minutes at 55% B. Compound detection was by UV absorbance at 280 nm.

Analytical Method C

The HPLC system comprised an Agilent HP1100 and was performed on 3 micron BDS C18 Hypersil (ThermoHypersil-Keystone Ltd) column, 150×4.6 mm, maintained at 40° C., running a mobile phase of:
Mobile phase A: deionized water.
Mobile phase B: acetonitrile.
Flow rate: 1 mL/minute.
This system was coupled to a Bruker Daltonics Esquire3000 electrospray mass spectrometer. Positive negative switching was used over a scan range of 500 to 1000 Dalton.
A linear gradient from 55% B-95% B was used over 10 minutes, followed by 2 minutes at 95% B, 0.5 minutes to 55% B and a further 2.5 minutes at 55% B.

Analytical Method D

Injection volume: 0.025 mL. HPLC was performed on 3 micron Gemini NX C18 (Phenomenex) column, 150×4.6 mm, maintained at 50° C., running a mobile phase of:

Mobile phase A: deionized water with formic acid (0.1%)

Mobile phase B: acetonitrile with formic acid (0.1%)

Flow rate: 1 mL/minute.

A linear gradient from 55% B-95% B was used over 10 minutes, followed by 2 minutes at 95% B, 0.5 minutes to 55% B and a further 2.5 minutes at 55% B. Compound detection was by UV absorbance at 280 nm.

Analytical Method E

Mobile Phase A 10 mM Ammonium Acetate/Water

Mobile Phase B ACN

Column FluoroSep-RP Phenyl HS, 50×2.1 mm, 5 □m

Column temperature Ambient

Autosampler needle washing soln 0.5% Formic Acid in 10% ACN/Water

Injection volume 0.012 ml

Autosampler temperature 10° C.

WYE-126657 retention time 3.8 min.

IS (WAY-130779) retention time 3.8 min.

Total run time 6.7 min.

Gradient Program

| Total | | | | |
|---|---|---|---|---|
| Time | Flow Rate | Mobile Phase | | Mobile Phase |
| (min) | (mL/min) | A (%) | B (%) | Gradient Profile |
| 0.0 | 0.6 | 90 | 10 | 1.0 |
| 1.5 | 0.6 | 90 | 10 | 1.0 |
| 3.5 | 0.6 | 10 | 90 | 1.0 |
| 5.0 | 0.6 | 10 | 90 | 1.0 |
| 5.2 | 0.6 | 90 | 10 | 1.0 |
| 6.7 | 0.6 | 90 | 10 | 1.0 |

Mass Spectrometry Conditions

### Sciex API 4000 (Serial No.: V09300509) Batman

Experiment: MRM (multiple reaction monitoring)

| Ion | | | | | | | | |
|---|---|---|---|---|---|---|---|---|
| Source | Curtain | CAD | GS1 | GS2 | IS | Temp (° C.) | Ihe | EP |
| Turbo | 25 | 4 | 50 | 50 | +5000 | 500 | on | 10 |
| Spray | | | | | | | | |

| | MS/MS | | Dwell | | | |
|---|---|---|---|---|---|---|
| Compound | Transition* | MRM | (msec) | DP | CE | CXP |
| 37R-hydroxynor- | 913.7 > 864.6 | —NH₃ | 250 | 76 | 17 | 18 |
| bornylrapamycin | | CH₃OH | | | | |
| temsirolimus (IS) | 1047.6 > 980.6 | —NH₃ | 250 | 91 | 29 | 20 |
| | | —CH₃OH | | | | |
| | | —H₂O | | | | |
| Phosphatidylcholine | 184 > 184 | | 250 | 150 | 7 | 5 |
| (matrix effect) | | | | | | |

*(M+NH₄)+ is parent ion.

### Example 8

### Generation of S. hyqroscopicus BIOT-4010 or MG2-10

For methodology to generate S. hygroscopicus MG2-10, refer to Example 2 in WO 2004/007709 (US 2005/0272132A1]. This strain can be used in place of BIOT-4010 to generate the compound of Formula II, following transformation, using standard protocols, with a vector expressing rapIJMNOL, such as pLL158 (WO2006/016167 (US 2009/0253732A1), Gregory et al., 2012).

BIOT-3410 is a higher-producing derivative of the rapamycin-producing strain of S. hygroscopicus NRRL5491, generated by mutagenesis and selection of higher producing variants and BIOT-4010 is a mutant of BIOT-3410 in which rapK has been specifically deleted, using similar methodology to that described for S. hygroscopicus MG2-10. BIOT-4010 is therefore a higher producing variant of S. hygroscopicus MG2-10, based on the selected strain. However, S. hygroscopicus NRRL5491 itself, or a derivative, could be used to generate a strain capable of producing compounds of the invention.

A naturally occurring Mfel site exists close to the 5′-end of rapK. To generate upstream and downstream areas of homology for integration, the 7.3 kbp Nco l fragment from pR 19 (Schwecke et al., 1995) has been cloned into plitmus28 that had been digested with Nco l and dephosphorylated, and the 4.2 kbp Nhei/Pst l fragment from cosmid-2 (Schwecke et al., 1995) was cloned into plitmus28 digested with Pstl-Spel. This gave intermediate plasmids plitmus28-7.3 and plitmus28-4.2 respectively. To introduce the desired deletion from the Mfel site to an internal site of rapK two oligonucleotides were used to amplify the required region, BioSG159: 5′-CCCCAATTGGTGTCGCTCGAGAACATCGCCCGGGTGA-3′ (SEQ ID NO:1) and BioSG 158: 5′-CGCCGCAAGTAGCACCGCTCGGCGAAGATCTCCTGG-3′ (SEQ ID NO:2) using plasmid pR 19 as template (Schwecke 1995). The resulting 1.5 kbp PCR product was treated with T4 polynucleotide kinase and cloned into plitmus28 that had been digested with EcoRVand dephosphorylated, and the cloned PCR product was sequenced. The 1.5 kbp Mfei-Bg/11 fragment from this plasmid was excised and used to replace the 2.3 kbp Mfei-Bg/11 fragment of plitmus28-4.2. To complete the construct, the 3.3 kbp Mfei-Hindlll fragment of this plasmid was ligated into similarly digested plitmus28-7.3. Finally, the deletion construct was transferred into the conjugative Streptomyces vector pKC 1132 (Bierman et al., 1992) as a Hindlll/Xbal fragment. The final construct was designated pSG3998.

Plasmid pSG3998 was transformed by electroporation into E. coli ET12567:pUZ8002 and selected on 2TY plates containing apramycin (0.050 mg/mL), kanamycin (0.025 mg/mL) and chloroamphenicol (0.0125 mg/mL) which were incubated at 30° C. overnight. Colonies were used to inoculate liquid 2TY media (4 mL) containing the same antibiotics and incubated overnight at 30° C. and 250 rpm. Approximately 0.8 mL of overnight culture was used to inoculate 2TY (10 mL) containing the same antibiotics and incubated at 30° C. and 250 rpm until they reached an OD-0.5 (595 nm). Cultures were centrifuged at 4000 rpm, washed twice with 2TY and the resulting cell pellet was resuspended in 2TY (0.25 mL). Spores of BIOT-3401 were thawed and pelleted by centrifugation (4000 rpm) and washed with 2TY (1 mL) before suspending in 2TY (1 mL). Spores were then exposed to heat shock at 50° C. for 10 min before placing immediately on ice. Approximately 100 uL spore stock was used per conjugation, and 2TY (0.150 mL) was added to adjust the volume to 0.25 mL. Conjugations were performed by mixing 0.25 mL of the washed E. coli cells with the adjusted BIOT-3401 spore stock and spreading immediately on a dried R6 plate. Plates were dried briefly, wrapped in clingfilm and incubated at 37° C. for 2-3 h. Each plate was then overlaid with sterile water (1 mL) containing nalidixic acid (0.015 mL of a 50 mg/L solution), dried and incubated at 37° C. overnight. The plates were then overlaid with sterile water (1 mL) containing apramycin (0.015 mL of a 100 mg/L solution) and incubated at 37° C. Ex-conjugate colonies appeared after 4-7 days and were picked onto Medium 1 plates containing apramycin (0.050 mg/mL) and nalidixic acid (0.025 mg/mL), and incubated at 37° C. for 3-4 days before re-patching to Medium 1 plates containing apramycin (0.050 mg/mL) and nalidixic acid (0.025 mg/mL). This patching process was then repeated for three rounds on Medium 1 plates with no antibiotics, incubating at 37° C. until good growth was visible. The patches were then transferred to Medium 1 plates and incubated at 28° C. to encourage sporulation (˜7-10 days). Spores were harvested, filtered through cotton wool and dilution series prepared. Aliquots (100 uL) of the dilution series were plated onto Medium 1 plates and incubated at 28° C. until spores were visible on the colonies. Colonies were patched in parallel to plates with and without apramycin (0.050 mg/mL). Apramycin sensitive colonies, representing candidate secondary recombinants, were then grown to assess rapamycin production. Non-producers were tested further by addition of exogenous trans-4-hydroxyCHCA to the production media after 24 h to confirm rapalog mutasynthetic production and verify the desired disruption of rapK.

### Example 9

### Fermentation and Isolation of Formula II

Liquid Culture (Small Scale)

A plug of BIOT-4010 was used to inoculate RapV7 seed media (7 mL) in a Falcon tube (50 mL) plugged with a foam bung and cultured at 28° C. and 300 rpm (2.5 cm throw) for 48 hours. MD6 production media (7 mL) was inoculated with this seed culture (0.5 mL) using a wide-bore tip and fermented for 6 days at 26° C. and 300 rpm (2.5 cm throw). Formula III was added after 24 hours of growth in production media. Feeds can be prepared as a 0.32 M stock solution in methanol and 0.050 mL added to each tube to give a final concentration of 2 mM.

Fermentation (Preparative)

Seed Conditions

| | |
|---|---|
| vessel | 2 L Erlenmeyer flask (foam bung stoppered) |
| working volume | 400 mL |
| medium | RapV7 seed medium |
| inoculum | 0.2 mL spore stock (0.05% v/v) |
| temperature | 28° C. |
| agitation | 250 rpm, 2.5 cm throw |
| aeration | aerobic |

transfer~48 h

Fermentation Conditions

| | |
|---|---|
| vessel | 22 L Braun Biostat C Bioreactor |
| working volume | 15 L (final working volume, including seed) |
| medium | MD6/5-1 |
| inoculum | 400 mL seed (2.7% v/v) |
| temperature | 26° C. |
| agitation | [symbol] 200 rpm (cascade DO2control) |
| aeration | 7.5 L/min air (0.5 v/v/m) |
| pH | 6.40 base (NaOH) only control 0-24 h (pre-precursor) |
| | 6.90 acid (H2SO4) only control >24 h (post-precursor) |
| DO2 | [symbol] 30% (cascade agitation control) |
| antifoam | automatic, on demand (SAG471) |
| add d-fructose | after sterilization |
| add l-lysine | ~24 h (just before precursor) |
| add Formula III | ~24 h (2 mM final conc.) |

harvest peak compound titer, usually from 132 hours-144 hours

Extraction and Purification

The fermentation broth was clarified by centrifugation (3000 rpm, 30 min) and the supernatant discarded if containing less than 5% total material. The cell paste was suspended in acetonitrile (2 volumes) and stirred at room temperature for 1 h. The resulting slurry was centrifuged and the supernatant decanted. This procedure was repeated, the supernatants combined, and the acetonitrile removed under reduced pressure at 40° C. The resulting aqueous slurry was extracted twice with an equal volume of ethyl acetate, the organic fractions combined and the solvent removed under reduced pressure at 40° C. The resulting crude extract was analyzed for 37R-hydroxynorbornylrapamycin content and was stored at 4° C. prior to chromatographic separation.

The crude extract was dissolved in methanol:water (80:20; 200-300 mL) and extracted twice with an equal volume of hexane. The methanol:water phase was retained and solvent removed under reduced pressure at 40° C. to yield a viscous liquid residue. This material was chromatographed over flash silica gel (25×5 cm column) eluting first with chloroform (1 L) and then with volumes of 1 L each 1%, 2% and 3% methanol in chloroform. Fractions of ˜250 mL were taken and analyzed by HPLC. The solvent was removed from fractions containing BC319 to leave a solid residue. This was chromatographed further over flash silica gel (20×2.5 cm column) eluting with ethyl acetate:hexane (1:1). Fractions of ˜200 mL were taken and analyzed by HPLC. Fractions containing the peak equivalent to feeding stock were pooled and the solvent was removed to leave a solid residue. This was chromatographed over reversed-phase silica gel (Waters XTerra C18-ODS2, 10 micron particle size, 19×250 mm) eluting with a gradient of water (A) and acetonitrile (B) at a flow rate of 21 mL/min: T=0 min, 50% B; T=25 min, 100% B. Fractions containing the peak equivalent to feeding stock were pooled and the solvent removed in vacuo to yield the compound of Formula I.

While certain embodiments have been described in terms of the preferred embodiments, it is understood that variations and modifications will occur to those skilled in the art. Therefore, it is intended that the appended claims cover all such equivalent variations that come within the scope of the following claims.

1. A compound of Formula I: 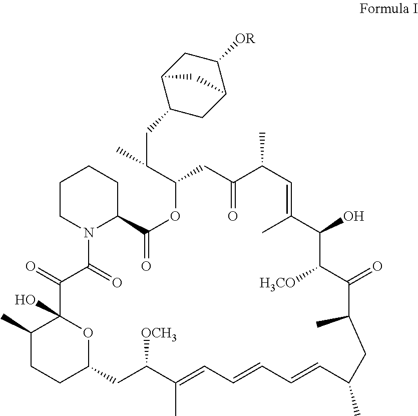
wherein:
R is selected from H or —C(O)(CR³R⁴)_{b}(CR⁵R⁶)_{d}(CR⁷R⁸R⁹);
R³ and R⁴ are each, independently, hydrogen, C₁ to C₆ alkyl, C₂ to C₈ alkenyl, C₂ to C₈ alkynyl, trihalomethyl, or —F;
R⁵ and R⁶ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹;
R⁷ is hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹;
R⁸ and R⁹ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹, or R⁸ and R⁹ can be taken together to form X or a cycloalkyl ring of 3-8 carbon atoms that is optionally mono-, di-, or tri-substituted with —(CR³R⁴)_{f}OR¹⁰;
R¹⁰ is hydrogen, C₁ to C₆ alkyl, C₂ to C₈ alkenyl, C₂ to C₈ alkynyl, tri-(C₁ to C₆ alkyl)silyl, tri-(C₁ to C₆ alkyl)silylethyl, triphenylmethyl, benzyl, C₂ to C₈ alkoxymethyl, tri-(C₁ to C₆ alkyl)silylethoxymethyl, chloroethyl, or tetrahydropyranyl;
R¹¹ is hydrogen, C₁ to C₆ alkyl, C₂ to C₈ alkenyl, C₂ to C₈ alkynyl, or a C₇ to C₁₀ phenylakyl;
X is 5-(2,2-di-(C₁ to C₆ alkyl)[1,3]dioxanyl, 5-(2,2-di-(C₃ to C₈ cycloalkyl)[1,3]dioxanyl, 4-(2,2-di-(C₁ to C₆ alkyl)[1,3]dioxanyl, 4-(2,2-di-(C₃ to C₈ cycloalkyl)[1,3]dioxanyl, 4-(2,2-di-(C₁ to C₆ alkyl)[1,3]dioxalanyl, or 4-(2,2-di-(C₃ to C₈ cycloalkyl)[1,3]dioxalanyl;
b is a whole number from 0 to 6;
d is a whole number from 0 to 6; and,
f is a whole number from 0 to 6; and/or,
a pharmaceutically acceptable salt, solvate, ester, or mixture thereof.

2. The compound and/or pharmaceutically acceptable salt, solvate, ester or mixture of claim 1 wherein R contains at least one moiety selected from H, —(CR³R⁴)_{f}OR¹⁰, X or —(CR³R⁴)_{f}OR¹⁰ substituted C₃ to C₈ cycloalkyl.

3. A composition comprising the compound of claim 1, or a pharmaceutically acceptable salt, solvate, ester or mixture thereof.

4. The composition of claim 3 further comprising a pharmaceutically acceptable carrier.

5. A compound of Formula II: 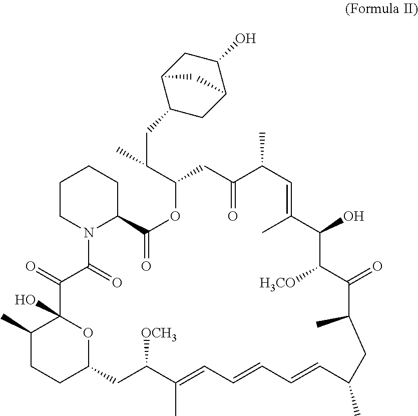 [SMILES: CO[C@H]1C[C@@H]2CC[C@@H](C)[C@@](O)(O2)C(=O)C(=O)N2CCCC[C@H]2C(=O)O[C@H]([C@H](C)C[C@H]2C[C@H]3C[C@@H]2C[C@@H]3O)CC(=O)[C@H](C)/C=C(\C)[C@@H](O)[C@@H](OC)C(=O)[C@H](C)C[C@H](C)/C=C/C=C/C=C/1C]
and/or a pharmaceutically acceptable salt, solvate, ester, or mixture thereof.

6. A composition comprising the compound of claim 5, or pharmaceutically acceptable salt, solvate, ester or mixture thereof.

7. The composition of claim 6 further comprising a pharmaceutically acceptable carrier.

8. A composition comprising about 70% or more of a compound selected from the group consisting of the compound of claim 5 (Formula II),
a pharmaceutically acceptable salt thereof, a solvate thereof, an ester thereof of the compound of formula (I), and mixtures of the foregoing.

9. The composition of claim 8, wherein the composition comprises about 90% or more of a compound selected from the group consisting of the compound of Formula (I), pharmaceutically acceptable salts and solvates of the compound of Formula (II), and mixtures of the foregoing.

10. The composition of claim 8, wherein the composition contains an essentially pure mixture of a compound selected from the group consisting of the compound of Formula (II), pharmaceutically acceptable salts, solvates, and esters of the compound of formula (I), and mixtures of the foregoing.

11. A pharmaceutical composition comprising a pharmaceutically acceptable carrier, diluent or excipient and one or more polyketides, wherein if there is one polyketide it is the polyketide of claim 5, and wherein if there is more than one polyketide in the composition then the polyketide of claim 5 comprises at least about 70% of the polyketide component of the pharmaceutical composition (Formula II)
wherein a pharmaceutically acceptable salt, solvate, and/or hydrate of the compound of claim 5 comprises at least 70% of the polyketide component of the pharmaceutical composition.

12. The pharmaceutical composition of claim 11, wherein the compound of Formula II is present in an amount of at least 80% of the polyketide component of the pharmaceutical composition.

13. The pharmaceutical composition of claim 11, wherein the compound of Formula II is present in an amount of at least 90% of the polyketide component of the pharmaceutical composition.

14. The pharmaceutical composition of claim 11, wherein the compound of Formula II is present in an amount of at least 95% of the polyketide component of the pharmaceutical composition.

15. The pharmaceutical composition of claim 11, wherein the compound of Formula II is present in an amount of at least 98% of the polyketide component of the pharmaceutical composition.

16. The pharmaceutical composition of claim 11, wherein the compound of Formula II is essentially the only polyketide in the pharmaceutical composition.

17. The composition of claim 3, wherein the solvate, if present, is a hydrate.

18. The composition of claim 3 wherein the composition is or comprises a bead, tablet, capsule, solution, or suspension.

19. A method of inhibiting the proliferation of a cell, the method comprising contacting said cell with an antiproliferative amount of a compound, pharmaceutically acceptable salt thereof, solvate thereof, ester thereof, or mixture thereof of any preceding claim; and/or the composition of claim 1.

20. The method of claim 19, wherein the cell is a human cell.

21. The method of claim 20, wherein the cell is a human cancer cell.

22. The method of claim 21, wherein the human cancer cell is selected from the group consisting of a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma.

23. The method of claim 19 wherein the method is an in vitro method.

24. The method of claim 19 wherein the method is an in vivo method.

25. A method of treating a mammal having a disease, the method comprising administering to said mammal an effective amount of a compound of claim 5, pharmaceutically acceptable salt thereof, solvate thereof, ester thereof, or mixture thereof and/or comprising the compound of claim 5; and/or a composition comprising claim 5.

26. The method of claim 25, wherein the mammal is human.

27. The method of claim 25, wherein the disease is cancer and the cancer is selected from the group consisting of a blood cancer, bone cancer, solid tumor, adenocarcinoma, brain cancer, glioblastoma, breast adenocarcinoma, bone marrow cancer, erythroleukemia, osteosarcoma, colorectal carcinoma, epidermoid carcinoma, epithelial carcinoma, uterine carcinoma, fibrosarcoma, gastric adenocarcinoma, kidney cancer, leukemia, acute lymphoblastic leukemia, chronic myelogenous leukemia, leiyomyoblastoma, lung carcinoma, small cell lung carcinoma, lymphoma, B cell lymphoma, Burkitt's lymphoma, T cell lymphoma, melanoma, malignant melanoma, neuroblastoma, leukemia ovarian cancer, ovary adenocarcinoma, pancreatic cancer, prostate adenocarcinoma, rhabdomyosarcoma, renal cell carcinoma, sarcoma, uterine sarcoma, squamous cell carcinoma, bladder squamous cell carcinoma, head and neck cancer, and transitional cell carcinoma.

28. The method of claim 25, wherein the compound of Formula I and/or Formula II administered as the sole active pharmaceutical agent(s); or the compound(s) of Formula I and/or Formula II is administered in combination with one or more of a chemotherapeutic agent, anti-cancer agent, or immune modulator; and/or radiation therapy and/or surgery.

29. The method of claim 25, wherein the mammal is in need of prevention of organ transplant rejection or host-versus-graft disease.

30. The method of claim 25 wherein the administration is via a route selected from the group consisting of parenteral, oral, topical, buccal, sublingual, transdermal, a medical device, a stent, inhalation, injection, subcutaneous, intramuscular, or intravenous; wherein the administration comprises a single dose or multiple doses at the same or different dosages; and/or the members of a combination are administered physically and/or temporally simultaneously or separately.

31. The method of claim 25 wherein the compound of Formula I and/or Formula II is provided as a bead, tablet, capsule, solution, or suspension.

32. A process for preparing a compound of Formula (I) according to claim 1 or a pharmaceutically acceptable salt thereof, the process comprising a feeding starter exo-(1R,2S,4R,5S)-5-hydroxybicyclo[2.2.1]heptane-2-carboxylate of formula (III): 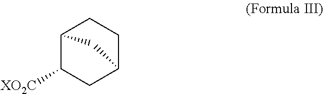
where X═H, alkyl, sodium or potassium, to a rapamycin-producing strain that has been genetically altered to remove or inactivate the rapK gene or a homologue thereof.

33. A prodrug of Formula II, wherein the prodrug is a polyketide of Formula I, 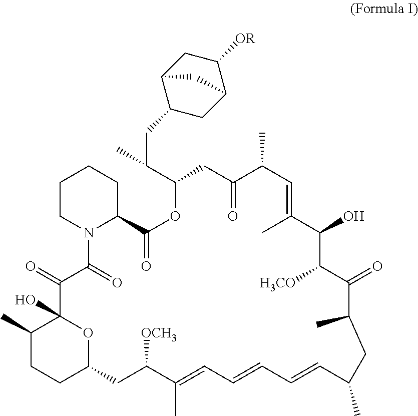
R is selected from H or —C(O)(CR³R⁴)_{b}(CR⁵R⁶)_{d}(CR⁷R⁸R⁹);
R³ and R⁴ are each, independently, hydrogen, C₁ to C₆ alkyl, C₂ to C₈ alkenyl, C₂ to C₈ alkynyl, trihalomethyl, or —F;
R⁵ and R⁶ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹;
R⁷ is hydrogen, C₁ to C₆ alkyl, C₂ to C₈ alkenyl, C₂ to C₈ alkynyl, —(CR³R⁴)_{f}OR¹⁰, —CF₃, —F, or CO₂R¹¹;
R⁸ and R⁹ are each, independently, hydrogen, C₁ to C₆ alkyl, C₂ to C₈ alkenyl, C₂ to C₈ alkynyl, —(CR³R⁴)_{f}OR¹⁰, —CF₃, —F, or CO₂R¹, or R⁸ and R⁹ can be taken together to form X or a cycloalkyl ring of 3-8 carbon atoms that is optionally mono-, di-, or tri-substituted with —(CR³R⁴)_{f}OR¹⁰;
R¹⁰ is hydrogen, C₁ to C₆ alkyl, C₂ to C₈ alkenyl, C₂ to C₈ alkynyl, tri-(C₁ to C₆ alkyl)silyl, tri-(C₁ to C₆ alkyl)silylethyl, triphenylmethyl, benzyl, C₂ to C₈ alkoxymethyl, tri-(C₁ to C₆ alkyl)silylethoxymethyl, chloroethyl, or tetrahydropyranyl;
R¹¹ is hydrogen, C₁ to C₆ alkyl, C₂ to C₈ alkenyl, C₂ to C₈ alkynyl, or a C₇ to C₁₀ phenylakyl;
X is 5-(2,2-di-(C₁ to C₆ alkyl)[1,3]dioxanyl, 5-(2,2-di-(C₃ to C₈ cycloalkyl)[1,3]dioxanyl, 4-(2,2-di-(C₁ to C₆ alkyl)[1,3]dioxanyl, 4-(2,2-di-(C₃ to C₈ cycloalkyl)[1,3]dioxanyl, 4-(2,2-di-(C₁ to C₆ alkyl)[1,3]dioxalanyl, or 4-(2,2-di-(C₃ to C₈ cycloalkyl)[1,3]dioxalanyl;
b is a whole number from 0 to 6;
d is a whole number from 0 to 6; and,
f is a whole number from 0 to 6; and/or,
a pharmaceutically acceptable salt, solvate, ester, hydrate, or mixture thereof.

34. A composition of claim 3, wherein the prodrug of Formula I is substituted for the polyketide of Formula II.

35. A method of claim 25, wherein the prodrug of Formula I is substituted for the polyketide of Formula II.
